(19) United States
(12) Patent Application Publication
(10) Pub. No.: US 2004/0121386 A1
(43) Pub. Date: Jun. 24, 2004

(54) MKK7gamma1 nucleic acids and polypeptides

(76) Inventor: Laura Michael, Ann Arbor, MI (US)

Correspondence Address: WARNER-LAMBERT COMPANY, 2800 PLYMOUTH RD, ANN ARBOR, MI, 48105, US

(21) Appl. No.: 10/706,116

(22) Filed: Nov. 12, 2003

Related U.S. Application Data
(60) Provisional application No. 60/425,581, filed on Nov. 12, 2002.

(51) Int. Cl. C12Q 1/68; C07H 21/04; C12N 9/12

(52) U.S. Cl. 435/6; 435/69.1; 435/194; 435/320.1; 435/325; 530/388.22; 536/23.2

(57) ABSTRACT

The invention relates to Mitogen-activated protein kinase kinase 7 (MKK7γ1) nucleic acids and amino acid sequences and methods of assaying for MKK7γ1 activity.

### CROSS-REFERENCES TO RELATED APPLICATIONS

This application claims the benefit of U.S. Provisional Patent application Serial No. 60/425,581, filed on Nov. 12, 2002, the teachings of which are herein incorporated by reference.

### BACKGROUND OF THE INVENTION

Mitogen-activated protein kinase (MAPK) pathways are activated by various extracellular stimuli including growth factors, cell stress, and proinflammatory cytokines. A canonical MAPK pathway consists of a MEKK that phosphorylates and activates an MKK which in turn phosphorylates and activates a MAPK. Activation of MAPKs ultimately leads to activation of transcription factors thereby regulating gene expression.

Three of the most well-characterized MAPK pathways are the ERK, JNK, and p38 pathways. MKKs are dual-specificity kinases that phosphorylate their respective MAPKs on a threonine and tyrosine residue (Pearson et al. Endocrine Rev., 2001;22(2):153-183; Kyriakis and Avruch J. Physiol. Rev., 2001;81(2):808-869).

The mitogen-activated protein kinase kinases 7 (MKK7) are a family of protein kinases that function as upstream activators of the mitogen-activated protein kinases (MAPKs). A MKK7 becomes activated in response to cellular stress, and it in turn activates the MAPK c-Jun N-terminal kinase (JNK) via phosphorylation on a threonine and tyrosine residue in a Thr-Pro-Tyr motif within kinase subdomain VIII.

The MKK7 class of MKKs are one of two identified upstream activators of JNK, along with MKK4. MKK7s are activated by various stimuli including UV, hyperosmolarity, LPS, TNF-α, and IL-3 (Tournier et al. Mol. Cell. Biol., 1999;19(2):1569-1581; Foltz et al. J. Biol. Chem., 1998;273(15):9344-9351). Studies in MKK7 gene knockout cells indicate a requirement for MKK7 in optimal activation of JNK following TNF-α and IL-1α stimulation (Dong et al. Nature, 2000;405: 91-94; Tournier et al. Genes and Dev., 2001; 15:1419-1426). Members of the MKK7 family are alternatively spliced; 6 different murine and 3 different human isoforms of MKK7 have been described in the literature (Tournier et al. Mol. Cell. Biol., 1999;19(2):1569-1581; Foltz et al. J. Biol. Chem., 1998;273(15):9344-9351). Tournier et al. describe the isolation and characterization of human MKK7α and MKK7β as well as several murine isoforms from a mouse testis cDNA library using mouse MKK7α1 as a probe. (Tournier et al. Mol. Cell. Biol., 1999;19:1569-1581). The murine isoforms include MKK7α2, MKK7β1, MKK7β2, MKK7γ1, and MKK7γ2 (Tournier et al. Mol. Cell. Biol., 1999;19(2):1569-1581).

To date, at least 6 different isoforms of murine MKK7γ1 have been identified. However, only 3 isoforms of human MKK7γ1 have been reported. To date there has been no report of a human homolog to the murine MKK7γ1 isoform (see Foltz et al., supra., 1998). Inhibitors of members of the MAPK pathways are being developed for the treatment of human disease (see e.g., English and Cobb. TIPS, 2002;23:40-45; Kumar and Madison. Expert Opin. Emerging Drugs, 2001;6:303-315). Indeed, companies have advanced MAPK pathway enzyme inhibitors into clinical trials for the treatment of inflammatory diseases and cancer (English and Cobb, supra., 2002). Therefore, there is a need in the art for the identification of novel MAPK pathway enzymes for the diagnosis of human disease and to aid in the identification of inhibitors of MAPK pathway enzymes and effectors.

### SUMMARY OF THE INVENTION

In one aspect, the present invention provides for an isolated nucleic acid comprising a nucleotide sequence encoding a 435 amino acid polypeptide of SEQ ID NO: 17, wherein X²⁸⁷ is Asp, Glu, or Ser; wherein X²⁹¹ is Asp, Glu, or Thr; and wherein X²⁹³ is Asp, Glu, or Ser. In certain embodiments, the isolated nucleic acid encodes a polypeptide selected from the group consisting of: SEQ ID NO: 2, SEQ ID NO: 4, SEQ ID NO: 6, SEQ ID NO: 8, SEQ ID NO: 10, and SEQ ID NO: 23. In other embodiments, the isolated nucleic acid comprises a nucleic acid selected from the group consisting of: SEQ ID NO: 1, SEQ ID NO: 3, SEQ ID NO: 5, SEQ ID NO: 7, SEQ ID NO: 9, and SEQ ID NO: 22. In still other embodiments, the isolated nucleic acid consists of a nucleotide acid selected from the group consisting of: SEQ ID NO: 1, SEQ ID NO: 3, SEQ ID NO: 5, SEQ ID NO:7, SEQ ID NO:9, and SEQ ID NO: 22.

In another aspect, the present invention provides for an isolated polypeptide comprising a 435 amino acid polypeptide of SEQ ID NO: 17, wherein X²⁸⁷ is Asp, Glu, or Ser; wherein X²⁹¹ is Asp, Glu, or Thr; and wherein X²⁹³ is Asp, Glu, or Ser. In certain embodiments, the isolated polypeptide comprises a polypeptide selected from the group consisting of: SEQ ID NO: 2, SEQ ID NO: 4, SEQ ID NO: 6, SEQ ID NO: 8, SEQ ID NO: 10, and SEQ ID NO: 23. In other embodiments, the isolated polypeptide consists of a polypeptide selected from the group consisting of: SEQ ID NO: 2, SEQ ID NO: 4, SEQ ID NO: 6, SEQ ID NO:, SEQ ID NO: 10, and SEQ ID NO: 23.

In yet another aspect, the invention relates to an expression cassette comprising: a promoter operably linked to a nucleic acid encoding the 435 amino acid polypeptide of SEQ ID NO: 17, wherein X²⁸⁷ is Asp, Glu, or Ser; wherein X²⁹¹ is Asp, Glu, or Thr; and wherein X²⁹³ is Asp, Glu, or Ser. In certain embodiments, the nucleic acid encodes a polypeptide selected from the group consisting of: SEQ ID NO: 2, SEQ ID NO: 4, SEQ ID NO: 6, SEQ ID NO: 8, SEQ ID NO: 10, and SEQ ID NO: 23. In other embodiments, the nucleic comprises a nucleic acid selected from the group consisting of: SEQ ID NO: 1, SEQ ID NO: 3, SEQ ID NO: 5, SEQ ID NO: 7, SEQ ID NO: 9, and SEQ ID NO: 22. In still other embodiments, the nucleic acid consists of a nucleotide acid selected from the group consisting of: SEQ ID NO: 1, SEQ ID NO: 3, SEQ ID NO: 5, SEQ ID NO: 7, SEQ ID NO: 9, and SEQ ID NO: 22.

In still another aspect, the invention provides for a cell that comprises an expression cassette comprising a promoter operably linked to a nucleic acid encoding a 435 amino acid polypeptide of SEQ ID NO: 17, wherein X²⁸⁷ is Asp, Glu, or Ser; wherein X²⁹¹ is Asp, Glu, or Thr; and wherein X²⁹³ is Asp, Glu, or Ser. In certain embodiments, polypeptide selected from the group consisting of: SEQ ID NO: 2, SEQ ID NO: 4, SEQ ID NO: 6, SEQ ID NO: 8, SEQ ID NO: 10, and SEQ ID NO: 23. In other embodiments, the nucleic acid comprises SEQ ID NO: 1, SEQ ID NO: 3, SEQ ID NO: 5, SEQ ID NO: 7, SEQ ID NO: 9, or SEQ ID NO: 22. In still other embodiments, the nucleic acid is selected from the group consisting of: SEQ ID NO: 1, SEQ ID NO: 3, SEQ ID NO: 5, SEQ ID NO: 7, and SEQ ID NO: 9, and SEQ ID NO: 22. In certain embodiments, the cell is a eukaryotic cell or an E. coli cell.

In a further aspect, the invention provides for method for assaying protein kinase activity comprising: incubating an isolated polypeptide comprising: the 435 amino acid polypeptide of SEQ ID NO: 17, wherein X²⁸⁷ is Asp, Glu, or Ser; wherein X²⁹¹ is Asp, Glu, or Thr; and wherein X²⁹³ is Asp, Glu, or Ser; in the presence of ATP and a MKK7γ1 substrate; and determining whether said MKK7γ1 substrate is phosphorylated. In certain embodiments, the ATP is γ-³²P-ATP. In other embodiments, the isolated polypeptide comprises SEQ ID NO: 2, SEQ ID NO: 4, SEQ ID NO: 6, SEQ ID NO: 8, SEQ ID NO: 10, or SEQ ID NO: 23. In still other embodiments, the method further comprises incubating said isolated polypeptide in the presence of a candidate therapeutic agent having a molecular weight of between 100 Da and 1000 Da. In other embodiments, the MKK7γ1 substrate is JNK1.

### BRIEF DESCRIPTION OF THE DRAWINGS

FIG. 1 depicts nucleotide sequence of a human MKK7γ1 nucleic acid (SEQ ID NO: 1) (top line) and the amino acid sequence encoded by SEQ ID NO: 1 (SEQ ID NO: 2) (lower line).

FIG. 2 depicts an alignment of nucleotide sequences for a MKK7γ1 nucleic acid (top nucleotide sequence; SEQ ID NO: 1), and a murine MKK7γ1 nucleic acid sequence (bottom nucleotide sequence; SEQ ID NO: 11).

FIG. 3 depicts an alignment of amino acid sequences for a MKK7γ1 polypeptide (SEQ ID NO: 2), and a mouse MKKγ1 polypeptide (SEQ ID NO: 12).

### DEFINITIONS

As used herein, the following terms have the meanings ascribed to them unless specified otherwise.

A “candidate therapeutic agent is able to inhibit MKK7γ1 activity” if the agent can decrease one or more MKK7γ1 activities.

A “cell expresses MKK7γ1 above basal levels” when the cell produces MKK7γ1 protein or mRNA in amounts greater than amounts produced in the parent cell or the untransfected cell. The amount of MKK7γ1 protein or mRNA can be determined using methods known in the art, such as Western blots or Northern blots, respectively.

“MKK7γ1 activity” can include, but is not limited to a MKK7γ1 mediated-cellular event, MKK7γ1 protein kinase activity, MKK7γ1 modulations of gene transcription, or MKK7γ1 effects on IL-8 or cytokine production. These activities can be assayed by comparing the effect of the candidate therapeutic agents on a MKK7γ1 polypeptide or on a cell that recombinantly expresses a MKK7γ1 polypeptide to a control.

An “expression cassette” is a nucleic acid construct, generated recombinantly or synthetically, with a series of specified nucleic acid elements that permit transcription of a particular nucleic acid in a host cell. The expression cassette can be part of a plasmid, virus, or nucleic acid fragment. Typically, the expression cassette includes a nucleic acid to be transcribed operably linked to a promoter.

The term “transfect” or “transduce” refers to any manner of introducing a nucleic acid into a cell including electroporation, biolistics, injection, plasmid transfection, lipofection, viral transduction, lipid-nucleic acid complexes, naked DNA, etc. A “host cell” is a naturally occurring cell or a transformed cell that contains an expression cassette and supports the replication or expression of the expression cassette. Host cells may be cultured cells, explants, cells in vivo, and the like. Host cells may be prokaryotic cells such as E. coli, or eukaryotic cells such as yeast, insect, amphibian, or mammalian cells such as CHO, CV-1, COS cells, and the like.

“Tumorigenic sample” as used herein is a sample of biological tissue or fluid that contains nucleic acids or polypeptides of MKK7γ1. The biological tissue comprises cancer cells, transformed cells, a tumor, a tumor cell and the like. The fluid typically comprises cancer cells, transformed cells, a tumor, tumor cells and the like. Such samples include, but are not limited to, tissue isolated from humans, mice, and rats. Tumorigenic samples may also include sections of tissues such as frozen sections taken for histological purposes. A tumorigenic sample is typically obtained from a mammal such as a rat, mouse, cow, dog, guinea pig, or rabbit, and most preferably a primate such as chimpanzees or humans.

“Tumor cell” refers to precancerous, cancerous, and normal cells in a tumor.

“Cancer cells,” “transformed” cells, or “transformation” in tissue culture, refers to spontaneous or induced phenotypic changes that do not necessarily involve the uptake of new genetic material. Although transformation can arise from infection with a transforming virus and incorporation of new genomic DNA, or uptake of exogenous DNA, it can also arise spontaneously or following exposure to a carcinogen, thereby mutating an endogenous gene. Transformation is associated with phenotypic changes, such as immortalization of cells, aberrant growth control, and/or malignancy (see, Freshney, Culture of Animal Cells a Manual of Basic Technique, 3rd ed., 1994).

“Inhibitors” of MKK7γ1 or “MKK7γ1 inhibitory compounds” are compounds that decrease MKK7γ1 protein kinase activity or another MKK7γ1 activity, e.g., antagonists, ligands, etc. Samples or assays comprising MKK7γ1 that are treated with a potential inhibitor are compared to control samples without the inhibitor.

The term “recombinant” when used with reference, e.g., to a cell, or nucleic acid, protein, or vector, indicates that the cell, nucleic acid, protein or vector, has been modified by the introduction of a heterologous nucleic acid or protein or by the alteration of a native nucleic acid or protein, or that the cell is derived from a cell so modified. Thus, for example, recombinant cells express genes that are not found within the native (nonrecombinant) form of the cell or over-express genes that are found within the native form of the cell.

A “promoter” is defined as an array of nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription.

A “constitutive” promoter is a promoter that is active under most environmental and developmental conditions. An “inducible” promoter is a promoter that is active under specific environmental or developmental conditions.

The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

The terms “isolated,” “purified,” or “biologically pure” refer to material that is substantially or essentially free from components which normally accompany it as found in its native state. Purity and homogeneity are typically determined using analytical chemistry techniques such as polyacrylamide gel electrophoresis or high performance liquid chromatography. In particular, an isolated MKK7γ1 nucleic acid is separated from open reading frames that flank the MKK7γ1 gene and encode proteins other than MKK7γ1. The term “purified” denotes that a preparation of a specified protein is at least 85% (w/v), more preferably at least 95% (w/v), and most preferably at least 99% (w/v) of the preparation.

“Nucleic acid” refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, peptide-nucleic acids (PNAs). Unless otherwise indicated, a particular nucleic acid sequence also implicitly encompasses conservatively modified variants thereof (e.g., degenerate codon substitutions) and complementary sequences, as well as the sequence explicitly indicated.

The term “nucleic acid” is used interchangeably with gene, cDNA, mRNA, oligonucleotide, and polynucleotide.

The terms “polypeptide,” “peptide,” and “protein” are used interchangeably herein to refer to a polymer of amino acid residues. The terms apply to amino acid polymers in which one or more amino acid residue is an analog or mimetic of a corresponding naturally occurring amino acid, as well as to naturally occurring amino acid polymers.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., on a carbon that is bound to a hydrogen, a carboxyl group, an amino group, and an R group (e.g., homoserine, norleucine, methionine sulfoxide, methionine methyl sulfonium). Such analogs have modified R groups (e.g., norleucine) or modified peptide backbones, but retain the same basic chemical structure as a naturally occurring amino acid. Amino acid mimetics refer to chemical compounds that have a structure that is different from the general chemical structure of an amino acid, but that function in a manner similar to a naturally occurring amino acid.

Amino acids may be referred to herein by either their commonly known 3-letter symbols or by the 1-letter symbols recommended by the IUPAC-IUB Biochemical Nomenclature Commission. Nucleotides, likewise, may be referred to by their commonly accepted single-letter codes.

The term “identity” in the context of 2 nucleic acids or polypeptides, refers to the number of identical residues that a first contiguous sequence has when compared and aligned for maximum correspondence to a second contiguous sequence, as measured using a sequence comparison software program or by visual inspection.

For sequence comparison, typically one sequence acts as a reference sequence, to which test sequences are compared. When using a computerized sequence comparison algorithm, test and reference sequences are input into a computer, subsequence coordinates are designated, if necessary, and sequence algorithm program parameters are designated if changes from the default parameters are desired. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math., 1981;2:482, by the homology alignment algorithm of Needleman & Wunsch, J. Mol. Biol., 1970;48:443, by the search for similarity method of Pearson & Lipman, Proc. Nat'l. Acad. Sci. USA., 1988;85:2444), by computerized implementations of these algorithms (GAP, BESTFIT, FASTA, and TFASTA in the Wisconsin Genetics Software Package, Genetics Computer Group, 575 Science Dr., Madison, Wis.), GeneDoc Program, Version 2.6.001, or by visual inspection (see generally, Current Protocols in Molecular Biology, Ausubel et al., eds., 1998). Another example of an algorithm that is suitable for determining percent sequence identity and sequence similarity is the BLAST algorithm, which is described in Altschul et al., J. Mol. Biol., 1990;215:403-410. Software for performing BLAST analyses is publicly available through the National Center for Biotechnology Information website. Default program parameters can be used, or alternative parameters can be designated. The sequence comparison algorithm then calculates the percent sequence identities for the test sequences relative to the reference sequence, based on the program parameters.

The phrase “MKK7γ1-mediated disorder or condition” refers to a pathology or disease caused or exacerbated by a MKK7γ1 activity. Examples of MKK7γ1-mediated disorder or condition include, but are not limited to, an inflammatory disorder or disease, COPD, rheumatoid arthritis, osteoarthritis, and cancer.

The term “mammal” refers to a member of the class Mammalia. Examples of mammals include, without limitation, humans, primates, chimpanzees, rodents, mice, rats, rabbits, sheep, and cows.

The phrase “MKK7γ1-mediated gene expression” refers to transcription of a polynucleotide that is controlled, regulated, or modulated by a MKK7γ1 polypeptide.

A “reporter gene construct” is a polynucleotide comprised of a response element, a promoter and a reporter gene. The response element is operably linked to the promoter and the promoter is operably linked to the reporter gene. The “reporter gene” is a nucleic acid that encodes for a protein or enzyme that can be detected. Examples of reporter genes include, without limitation, luciferase, alkaline phosphatase, beta-galactosidase, chloramphenicol acetyltransferase, and the green fluorescent protein (GFP).

A “response element” is a polynucleotide sequence that has the ability to bind a transcription factor.

### DETAILED DESCRIPTION

I. Introduction

The present invention relates to MKK7γ1 nucleic acids and proteins, methods for isolating MKK7γ1 nucleic acids, methods for expressing MKK7γ1 proteins, and methods for the use of MKK7γ1 proteins and nucleic acids. Moreover, MKK7γ1 polypeptides can be used to assay for inhibitors of MKK7γ1. Such inhibitors are useful for pharmacological and genetic modulation of phenomena such as inflammatory processes and disorders, COPD, rheumatoid arthritis, osteoarthritis, and cancer. The invention thus also provides MKK7γ1 assays, including protein kinase assays, gene reporter assays, and biological assays for MKK7γ1 function. In addition, the present invention provides for the use of MKK7γ1 nucleic acid sequences in determining the presence of MKK7γ1 sequences in biological samples.

II. Isolation of the Encoding MKK7γ1 Nucleic Acids

A. General Recombinant DNA Methods

MKK7γ1 polypeptides and nucleic acids are used in the assays described below. For example, recombinant MKK7γ1 can be used to make cells that constitutively express MKK7γ1. Such polypeptides and nucleic acids can be made using routine techniques in the field of recombinant genetics. Basic texts disclosing the general methods of use in this invention include Sambrook et al., Molecular Cloning, A Laboratory Manual, 2nd ed., 1989; Kriegler, Gene Transfer and Expression: A Laboratory Manual, 1990; and Current Protocols in Molecular Biology, Ausubel et al., eds., 1998.

For nucleic acids, sizes are given in either kilobases (kb) or base pairs (bp). These are estimates derived from agarose or acrylamide gel electrophoresis, from sequenced nucleic acids, or from published DNA sequences. For proteins, sizes are given in kilodaltons (kDa) or amino acid residue numbers. Proteins sizes are estimated from gel electrophoresis, from sequenced proteins, from derived amino acid sequences, or from published protein sequences.

B. Isolation of MKK7γ1 Nucleic Acids Sequences

In general, the nucleic acid sequences encoding genes of interest, such as MKK7γ1 and related nucleic acid sequence homologs, are cloned from cDNA and genomic DNA libraries by hybridization with a probe, or isolated using amplification techniques with oligonucleotide primers. Preferably mammalian, more preferably human sequences are used. For example, MKK7γ1 sequences are typically isolated from mammalian nucleic acid (genomic or cDNA) libraries by hybridizing with a nucleic acid probe, the sequence of which can be derived from SEQ ID NO: 1.

A suitable tissue from which human MKK7γ1 RNA and cDNA can be isolated is skeletal muscle. Amplification techniques using primers can also be used to amplify and isolate, e.g., a nucleic acid encoding MKK7γ1, from DNA or RNA (see, e.g., Dieffenbach & Dveksler, PCR Primer: A Laboratory Manual, Cold Spring Harbor Laboratory, 1995). These primers can be used, e.g., to amplify either an MKK7γ1 nucleic acid sequence or a probe of one to several hundred nucleotides, which is then used to screen a mammalian library for the MKK7γ1 nucleic acid sequence (e.g., SEQ ID NO: 1). For example, degenerate primer pairs that correspond to amino sequences contained in SEQ ID NO: 2 can be used to isolate a MKK7γ1 nucleic acid.

To make a cDNA library, one should choose a source that is rich in the mRNA of choice, e.g., for human MKK7γ1 mRNA, human skeletal muscle tissue. The mRNA is then made into cDNA using reverse transcriptase, ligated into a recombinant vector, and transfected into a recombinant host for propagation, screening and cloning. Methods for making and screening cDNA libraries are well-known (see, e.g., Gubler & Hoffman, Gene, 1983;25:263-269; Sambrook et al., supra., 1989; Ausubel et al., supra., 1998).

For a genomic library, the DNA is extracted from the tissue and either mechanically sheared or enzymatically digested to yield fragments of about 12 to 20 kb. The fragments are then separated by gradient centrifugation from undesired sizes and are constructed in non-lambda expression vectors. These vectors are packaged in vitro. Recombinant phage are analyzed by plaque hybridization as described in Benton & Davis, Science, 1977;196:180-182. Colony hybridization is carried out as generally described in Grunstein et al., Proc. Nat'l Acad. Sci. USA, 1975;72:3961-3965.

An alternative method of isolating a nucleic acid and its homologs combines the use of synthetic oligonucleotide primers and amplification of an RNA or DNA template (see U.S. Pat. Nos. 4,683,195 and 4,683,202; (see e.g., Innis et al. eds., PCR protocols: A guide to methods and applications. Academic Press, Inc., 1990;21:21-27). Methods such as the polymerase chain reaction (PCR) and ligase chain reaction (LCR) can be used to amplify nucleic acid sequences of, e.g., MKK7γ1 directly from mRNA, from cDNA, from genomic libraries or cDNA libraries.

Polymerase chain reaction or other in vitro amplification methods may also be useful, for example, to clone nucleic acid sequences that code for proteins to be expressed, to make nucleic acids to use as probes for detecting the presence of MKK7γ1 encoding mRNA in physiological samples, for nucleic acid sequencing, or for other purposes. Genes amplified by the PCR reaction can be purified from agarose gels and cloned into an appropriate vector.

As described above, gene expression of MKK7γ1 nucleic acid sequences can also be analyzed by techniques known in the art, e.g., reverse transcription and PCR amplification of mRNA, isolation of total RNA or poly A+ RNA, northern blotting, dot blotting, in situ hybridization, RNase protection, probing oligonucleotide arrays, and the like.

Synthetic oligonucleotides can be used to construct recombinant genes for use as probes or for expression of protein. This method is performed using a series of overlapping oligonucleotides usually 40 to 120 bp in length, representing both the sense and nonsense strands of the gene. These DNA fragments are then annealed, ligated, and cloned. Alternatively, amplification techniques can be used with precise primers to amplify a specific subsequence of a MKK7γ1 nucleic acid. The specific subsequence is then ligated into an expression vector.

The nucleic acid encoding the protein of choice is typically cloned into intermediate vectors before transformation into prokaryotic or eukaryotic cells for replication and/or expression. These intermediate vectors are typically prokaryote vectors, e.g., plasmids, or shuttle vectors. Optionally, cells can be transfected with a recombinant MKK7γ1 operably linked to a constitutive promoter, to provide MKK7γ1 expression in cultured cells.

Mutant MKK7γ1 Sequences

Because of the degeneracy of the genetic code, a large number of functionally equivalent nucleic acids can encode a given MKK7γ1 polypeptide sequence. For instance, the codons GCA, GCC, GCG, and GCU all encode the amino acid alanine. Thus, at every position where an alanine is specified by a codon, the codon can be altered to any of the corresponding codons described without altering the encoded polypeptide. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule.

In addition, degenerate codon substitutions may be achieved by generating sequences in which the third position of one or more selected (or all) codons is substituted with mixed-base and/or deoxyinosine residues (Batzer et al., Nucleic Acid Res., 1991;19:5081; Ohtsuka et al., J. Biol. Chem., 1985;260:2605-2608; Rossolini et al., Mol. Cell. Probes, 1994;8:91-98). In some embodiments, the nucleotide sequences that encode the enzymes are preferably optimized for expression in a particular host cell (e.g., yeast, mammalian, plant, fungal, and the like) used to produce the enzymes.

Kinase Activation Mutants

MAP kinase kinases are phosphorylated on Ser and Thr residues in subdomain XIII by upstream kinases (e.g., MEKK1). The replacement of these residues (e.g., Ser²⁸⁷ and Thr²⁹¹; or Ser²⁸⁷, Thr²⁹¹, and/or Ser²⁹³) in SEQ ID NO: 2 by the negatively charged amino acids Asp or Glu can generate MKKs that exhibit higher activity than unphosphorylated wild-type MKKs (see e.g., Holtmann et al., Mol. Cell Biol., 1999;19(10):6742-6753; Mansour et al., Science, 1994;256:966-970; and Raingeaud et al., Mol. Cell. Biol., 1996;16:1247-1255). Thus, the present invention includes the polypeptide that comprise a 435 amino acid polypeptide of SEQ ID NO: 17, wherein X²⁸⁷ is Asp, Glu, or Ser; and wherein X²⁹¹ is Asp, Glu, or Thr; MKK7γ1 polypeptides include polypeptides where Ser²⁸⁷ is replaced by Asp or Glu, and Thr²⁹¹ is replaced by Asp or Glu. Examples of these double mutants include, but are not limited to, SEQ ID NO: 3, SEQ ID NO: 5, SEQ ID NO: 7, and SEQ ID NO: 9.

The present invention also includes polypeptides of SEQ ID NO: 17, wherein X²⁸⁷ is Asp, Glu, or Ser; and wherein X²⁹¹ is Asp, Glu, or Thr; and wherein X²⁹³ is Asp, Glu, or Ser; MKK7γ1 polypeptides include polypeptides where Ser²⁸⁷ is replaced by Asp or Glu; Thr²⁹¹ is replaced by Asp or Glu; and wherein X²⁹³ is Asp, Glu, or Ser. SEQ ID NO: 17 can also give rise to a wild-type MKK7γ1 sequence, e.g., SEQ ID NO: 2. An example of a triple mutant is SEQ ID NO: 23 (MKK7γ1 E²⁸⁷E²⁹¹E²⁹³) which is encoded by SEQ ID NO: 22. Examples of triple mutants are set out in Table 1:

TABLE 1

| # | 287 | 291 | 293 |
|---|---|---|---|
| 1 | Glu | Glu | Glu |
| 2 | Glu | Glu | Asp |
| 3 | Glu | Asp | Glu |
| 4 | Glu | Asp | Asp |
| 5 | Asp | Asp | Asp |
| 6 | Asp | Asp | Glu |
| 7 | Asp | Glu | Asp |
| 8 | Asp | Glu | Glu |

These and other mutant MKK7γ1 nucleotide sequences can be generated using methods known in the art, such as site-directed mutation technology, PCR, LCR, ligation of overlapping oligonucleotides, etc.

III. MKK7γ1 Polypeptides

Structurally, MKK7γ1 nucleotide sequences encode polypeptides that place them in the MKK class of protein kinases. The molecular mass of MKK7γ1 (SEQ ID NO: 2) is calculated at about 47,940 Da. MKK7γ1 (e.g., SEQ ID NO: 2) encodes a protein kinase capable of phosphorylating the MKK7γ1 substrate JNK1. As used herein, the term “kinase” includes a protein or polypeptide that is capable of autophosphorylation and/or the phosphorylation of a different protein or polypeptide.

IV. Expression in Prokaryotes and Eukaryotes

To obtain high level expression of a cloned gene or nucleic acid, such as those cDNAs encoding MKK7γ1, one typically subclones MKK7γ1 into an expression vector that contains a strong promoter to direct transcription, a transcription/translation terminator, and if for a nucleic acid encoding a protein, a ribosome binding site for translational initiation.

A. Prokaryotic Expression

Suitable bacterial promoters are well-known in the art and described, e.g., in Sambrook et al., supra., 1989 and Ausubel et al., supra., 1998. Bacterial expression systems for expressing the MKK7γ1 protein are available in, e.g., E. coli, Bacillus sp., and Salmonella (Palva et al., Gene, 1983;22:229-235). Kits for such expression systems are commercially available. Eukaryotic expression systems for mammalian cells, yeast, and insect cells are well-known in the art and are also commercially available.

In certain embodiments, a fusion protein comprising a ligand binding domain (e.g., a poly-histidine tag) fused directly or through a linker region to a MKK7γ1 polypeptide can be expressed. Polynucleotide vectors that facilitate the expression of fusion proteins are commercially available (e.g., New England Biolabs, Invitrogen and Novagen). For example, a polyhistidine tag (e.g., MGSSHHHHHHSSG) (SEQ ID NO: 21) can be fused to a MKK7γ1 amino acid sequence by subcloning a MKK7γ1 sequence into a pET28a, b or c vector (Novagen, Madison, Wis.). The histidine tagged-MKK7γ1 polypeptide can be expressed in E. coli and purified over a nickel affinity column by eluting with imidazole or another suitable eluant.

B. Eukaryotic Expression

A variety of methods are known in the art for expressing a gene in eukaryotes (Ausubel et al., supra., 1998). These methods often achieve expression of a gene above basal levels of MKK7γ1 expression in the native cell.

Cells can be transfected with an expression cassette containing a MKK7γ1 nucleic acid of interest (e.g., SEQ ID NO: 1) and a promoter. The promoter used to direct expression of a heterologous nucleic acid depends on the particular application. The promoter is preferably positioned about the same distance from the heterologous transcription start site as it is from the transcription start site in its natural setting. As is known in the art, however, some variation in this distance can be accommodated without loss of promoter function. The promoter typically can also include elements that are responsive to transactivation, e.g., hypoxia responsive elements, Gal4 responsive elements, lac repressor responsive elements, and the like. The promoter can be constitutive or inducible, heterologous or homologous.

In addition to the promoter, the expression vector typically contains a transcription unit or expression cassette that contains all the additional elements required for the expression of the nucleic acid in host cells. A typical expression cassette thus contains a promoter operably linked, e.g., to the nucleic acid sequence encoding MKK7γ1, and signals required for efficient polyadenylation of the transcript, ribosome binding sites, and translation termination.

In addition to a promoter sequence, the expression cassette should also contain a transcription termination region downstream of the structural gene to provide for efficient termination. The termination region may be obtained from the same gene as the promoter sequence or may be obtained from different genes.

Expression vectors containing regulatory elements from eukaryotic viruses are typically used in eukaryotic expression vectors, e.g., SV40 vectors, papilloma virus vectors, and vectors derived from Epstein-Barr virus. Other exemplary eukaryotic vectors include pMSG, pAV009/A⁺, pMT010/A⁺, pMAMneo-5, baculovirus pDSVE, and any other vector allowing expression of proteins under the direction of the SV40 early promoter, SV40 later promoter, metallothionein promoter, murine mammary tumor virus promoter, Rous sarcoma virus promoter, polyhedrin promoter, or other promoters shown effective for expression in eukaryotic cells.

Some expression systems have markers that provide gene amplification such as thymidine kinase, hygromycin B phosphotransferase, and dihydrofolate reductase.

Alternatively, high yield expression systems not involving gene amplification are also suitable, such as using a baculovirus vector in insect cells, with a MKK7γ1 encoding sequence under the direction of the polyhedrin promoter or other strong baculovirus promoters.

Any of the well-known procedures for introducing foreign nucleotide sequences into host cells may be used. These include the use of calcium phosphate transfection, polybrene, protoplast fusion, electroporation, liposomes, microinjection, plasma vectors, viral vectors, and any of the other well-known methods for introducing cloned genomic DNA, cDNA, synthetic DNA, or other foreign genetic material into a host cell (see, e.g., Sambrook et al., supra., 1989). It is only necessary that the particular genetic engineering procedure used be capable of successfully introducing at least one gene into the host cell capable of expressing the protein of choice.

V. Purification of MKK7γ1 Polypeptides

If necessary, recombinant MKK7γ1 proteins can be purified for use in enzyme assays, and for use in generating MKK7γ1 antibodies. Recombinant MKK7γ1 can be purified from any suitable expression system, e.g., by expressing a MKK7γ1 protein in a host and then purifying the recombinant protein via affinity purification. In some embodiments, the recombinant protein is a fusion protein that contains a tag that facilitates purification, e.g., a polyhistidine tag.

The MKK7γ1 protein can be also separated from other proteins and components of the source material, (e.g., a recombinant cell) by standard separation techniques, e.g., column chromatography. Protein purification techniques are well-known in the art and include, but are not limited to, solubility fractionation (e.g., precipitation with ammonium sulfate, etc.), immunoprecipitation, centrifugation, ultracentrifugation, gel filtration chromatography, ion exchange chromatography, affinity chromatography, immunoaffinity chromatography, (see e.g., Scopes, Protein Purification: Principles and Practice, Springer-Verlag New York Inc., 1982). Typically one or more of these techniques can be used to enrich for or isolate a MKK7γ1 polypeptide. VI. Methods for Identifying MKK7γ1 Inhibitory Compounds

Methods for identifying MKK7γ1 inhibitory compounds are described below. Compounds that inhibit MKK7γ1 or a MKK7γ1 mediated process (e.g., cell growth) may be useful as pharmaceutical agents in the treatment of MKK7γ1-mediated disorders and conditions. MKK7γ1 assays are carried out in the presence or absence of a MKK7γ1 inhibitory compound and the amount of enzyme activity is compared for a determination of inhibitory activity of the MKK7γ1 inhibitory compound. Samples that are not contacted with a MKK7γ1 inhibitory compound are assigned a relative MKK7γ1 activity value of 100. Inhibition of MKK7γ1 activity is achieved when the MKK7γ1 activity in the presence of a MKK7γ1 inhibitory compound is less than the control sample (i.e., no inhibitory compound). Preferably, the MKK7γ1 activity value relative to the control is less than 90%, more preferably less than 50%, and still more preferably less than. The IC₅₀ of a compound is the concentration of compound that exhibits 50% of the control sample activity. Typically, candidate therapeutic agents are screened to identify MKK7γ1 inhibitory compounds which can be further tested for their ability to treat a MKK7γ1 mediated disorder or condition.

A. Candidate Therapeutic Agents.

A “candidate therapeutic agent” is a compound that is being tested for its usefulness in the treatment of a MKK7γ1-mediated disorder or condition, or is being tested in a ligand binding assay or a gene activation assay. Essentially any chemical compound can be screened as a MKK7γ1 candidate therapeutic agent in a MKK7γ1 assay to identify a MKK7γ1 inhibitory compound. The compounds tested as inhibitors of MKK7γ1 can be any small chemical compound, or a biological entity, such as a protein, sugar, nucleic acid or lipid. Alternatively, inhibitors can be genetically altered versions of MKK7γ1. Typically, test compounds will be small chemical molecules and peptides. The candidate therapeutic agent can be a naturally occurring compound, one that is artificially synthesized, or one that is made by a combination these methods. In certain embodiments, the candidate therapeutic agents are organic molecules under 1000 Da, more preferably under 500 Da, and still more preferably are between 500 and 100 Da, and pharmaceutically acceptable salts thereof.

It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs, Switzerland) and the like which can be used as candidate therapeutic agents in MKK7γ1 assays.

In some embodiments, assays can be used to screen a combinatorial library that contains a large number of potential therapeutic compounds (potential inhibitor compounds). In some embodiments, compounds dissolved in aqueous or organic (e.g., DMSO-based) solutions are screened. “Combinatorial chemical libraries” can be screened in one or more MKK7γ1 assays to identify those library members (particular chemical species or subclasses) that display a desired characteristic activity. The compounds thus identified can serve as conventional “lead compounds” or can themselves be used as potential or actual therapeutics.

A combinatorial compound library is a collection of diverse compounds that have been generated by either chemical synthesis or biological synthesis, by combining a number of chemical “building blocks” such as reagents. For example, a linear combinatorial compound library such as a polypeptide library is formed by combining a set of chemical building blocks (amino acids) in every possible way for a given compound length (i.e., the number of amino acids in a polypeptide compound). A plethora of compounds can be synthesized through such combinatorial mixing of chemical building blocks. The preparation and screening of combinatorial chemical libraries and other libraries of large numbers of compounds (e.g., over 1000 compounds) are well-known to those of skill in the art.

B. Protein Kinase Assays

Methods for assaying MKK7 activity have been described in the art (see e.g., Tournier et al., supra., 1997; and U.S. Pat. No. 6,136,596). Typically, a sample containing a MKK7γ1 protein are combined with ATP (e.g., γ-³²P-ATP) and a MKK7γ1 substrate (e.g., JNK1). The MKK7γ1 protein can be activated by phosphorylation by another kinase such as MEKK1. The assays can also contain a JNK substrate, e.g., c-Jun, which also can be considered a MKK7γ1 substrate. The reactions are then typically stopped with SDS-PAGE sample buffer and then electrophoresed on SDS-PAGE. The presence of phosphorylated MKK7γ1 substrate or of a JNK substrate can be visualized by autoradiography or by scintillation counting of the radioactive bands.

C. Reporter Gene Assays for MKK7γ1 Activity

Also, provided by the invention are methods for assaying candidate therapeutic agents in a reporter gene assay. MKK7 activates the JNK pathway in cells which is known to up-regulate AP-1 transcriptional activity (see Tournier et al., supra., 1997). The transcription from a plasmid containing AP1 response elements fused to a reporter gene (e.g., pTRE-luciferase) was activated by MKK7 (Tournier et al., supra., 1997).

A variety of reporter genes are known in the art and can be used in a reporter gene construct containing an AP1 response element, such as chloramphenicol acetyltransferase (CAT) and β-galactosidase (e.g., bacterial LacZ gene), the firefly luciferase gene (see, e.g., Cara et al. J. Biol. Chem., 1996;271:5393-5397), the green fluorescence protein (see, e.g., Chalfie et al. Science, 1994;263:802), secreted alkalkine phosphatase, alkaline phosphatase, and many others. Selectable markers which facilitate cloning of the vectors of the invention are optionally included. Sambrook, supra., 1989 and Ausubel, supra., 1998, provide an overview of selectable markers.

Standard transfection methods can be used to introduce a MKK7γ1 expression cassette and a MKK7γ1 reporter gene construct into host cells. For mammalian host cells, preferred transfection methods include, for example, calcium phosphate precipitation (Chen and Okayama, BioTechniques, 1988;6:632), DEAE-dextran, and cationic lipid-mediated transfection (e.g., Lipofectin) (see, e.g., Ausubel, supra., 1998).

The cell containing the MKK7γ1 expression cassette and the reporter gene construct are contacted with a candidate therapeutic agent for a suitable amount of time, e.g., for a period ranging from 0.5 to 48 hours. For example, a cell that contains a reporter gene construct and the MKK7γ1 polypeptide can be grown in the presence and absence of a candidate therapeutic agent. In addition, MKK7γ1 activity can be increased using a ligand that directly or indirectly activates MKK7γ1 activity or an activation mutant of MKK7γ1 (e.g., a MKK7γ1 with Ser²⁸⁷ to Asp²⁸⁷ and Thr²⁹¹ to Asp²⁹¹ mutations, etc.).

D. Assays for Changes in Cell Growth

The effects of a MKK7γ1 inhibitor compound on any changes in cell growth can be assessed by using a variety of in vitro and in vivo assays. The phrase “changes in cell growth” refers to any change in cell growth and proliferation characteristics in vitro or in vivo, such as formation of foci, anchorage independence, semi-solid or soft agar growth, cytotoxicity, changes in contact inhibition and density limitation of growth, loss of growth factor or serum requirements, changes in cell morphology, gaining or losing immortalization, gaining or losing tumor specific markers, ability to form or suppress tumors when injected into suitable animal hosts, and/or immortalization of the cell. Assays for changes in cell growth include, but are not limited to assessing a cancer or tumor cell's ability to grow on soft agar, changes in contact inhibition and density limitation of growth, changes in growth factor or serum dependence, changes in the level of tumor specific markers, changes in invasiveness into Matrigel®, changes in tumor growth in vivo, such as in transgenic mice, analysis of cell cycle, etc. (see e.g., Freshney, Culture of Animal Cells: A Manual of Basic Technique, 4th ed. Wiley-Liss, Inc., 2000), herein incorporated by reference. Compounds that exhibit the ability to decrease a tumor or cancer cell's activity in one or more of these assays as compared to a control (e.g., absence of a compound of the present invention), may be useful as therapeutics for treatment of cancer and other diseases involving cellular hyperproliferation. The compounds may be assayed alone or in combination with other anticancer compounds in one or more of the assays set out below or in a cancer agent screening assay that is known in the art.

VII. Pharmaceutical Compositions

Accordingly, the present invention provides pharmaceutical compositions comprising a pharmaceutically acceptable carrier or excipient and a candidate therapeutic agent. Candidate therapeutic agents which possess IC₅₀s for decreasing MKK7γ1 activity of about 100 μM or less can be particularly useful in the present compositions. More preferably, the candidate therapeutic agent will have an IC₅₀ of about 1 μM or less. Most preferably the compounds will have an IC₅₀ of about 0.01 μM or less. Preferably, the candidate therapeutic agents will cause a decrease in symptoms associated with an MKK7γ1-mediated disorder as measured quantitatively or qualitatively.

The present invention also provides pharmaceutical compositions comprising a pharmaceutically acceptable carrier or excipient and a MKK7γ1 inhibitory compound. A MKK7γ1 inhibitory compound can be formulated as a pharmaceutical composition in the form of a syrup, an elixir, a suspension, a powder, a granule, a tablet, a capsule, a lozenge, a troche, an aqueous solution, a cream, an ointment, a lotion, a gel, an emulsion, etc. Preferably, a compound of the present invention will cause a decrease in symptoms or a disease indicia associated with a MKK7γ1-mediated disorder as measured quantitatively or qualitatively.

For preparing pharmaceutical compositions from the compounds of the present invention, pharmaceutically acceptable carriers can be either solid or liquid. Solid form preparations include powders, tablets, pills, capsules, cachets, suppositories, and dispersible granules. A solid carrier can be one or more substances which may also act as diluents, flavoring agents, binders, preservatives, tablet disintegrating agents, or an encapsulating material.

In powders, the carrier is a finely divided solid which is in a mixture with the finely divided active component. In tablets, the active component is mixed with the carrier having the necessary binding properties in suitable proportions and compacted in the shape and size desired.

The powders and tablets contain from 1% to 95% (w/w) of the active compound. In certain embodiments, the active compound ranges from 5% to 70% (w/w). Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

For preparing suppositories, a low melting wax, such as a mixture of fatty acid glycerides or cocoa butter, is first melted and the active component is dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution.

Aqueous solutions suitable for oral use can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, and other well-known suspending agents.

Also included are solid form preparations which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampules. Also, the unit dosage form can be a capsule, tablet, cachet, or lozenge itself, or it can be the appropriate number of any of these in packaged form.

The quantity of active component in a unit dose preparation may be varied or adjusted from 0.1 mg to 1000 mg, preferably 1.0 mg to 100 mg, or from 1% to 95% (w/w) of a unit dose, according to the particular application and the potency of the active component. The composition can, if desired, also contain other compatible therapeutic agents.

Pharmaceutically acceptable carriers are determined in part by the particular composition being administered, as well as by the particular method used to administer the composition. Accordingly, there is a wide variety of suitable formulations of pharmaceutical compositions of the present invention (see, e.g., Gennaro et al., editors. Remington: The Science and Practice of Pharmacy. 20th ed., Lippincott Williams and Wilkins, 2000).

A compound of the present invention, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane nitrogen, and the like.

Formulations suitable for parenteral administration, such as, for example, by intravenous, intramuscular, intradermal, and subcutaneous routes, include aqueous and nonaqueous, isotonic sterile injection solutions, which can contain antioxidants, buffers, bacteriostats, and solutes that render the formulation isotonic with the blood of the intended recipient, and aqueous and nonaqueous sterile suspensions that can include suspending agents, solubilizers, thickening agents, stabilizers, and preservatives. In the practice of this invention, compositions can be administered, for example, by intravenous infusion, orally, topically, intraperitoneally, intravesically, or intrathecally. The formulations of compounds can be presented in unit-dose or multi-dose sealed containers, such as ampules and vials. Injection solutions and suspensions can be prepared from sterile powders, granules, and tablets of the kind previously described.

The dose administered to a subject, in the context of the present invention should be sufficient to effect a beneficial therapeutic response in the subject over time. The dose will be determined by the efficacy of the particular compound employed and the condition of the subject, as well as the body weight or surface area of the subject to be treated. The size of the dose also will be determined by the existence, nature, and extent of any adverse side effects that accompany the administration of a particular compound in a particular subject. In determining the effective amount of the compound to be administered in the treatment or prophylaxis of the disorder being treated, the physician can evaluate factors such as the circulating plasma levels of the compound, compound toxicities, and/or the progression of the disease, etc. In general, the dose equivalent of a compound is from about 1 μg/kg to 10 mg/kg for a typical subject. Many different administration methods are known to those of skill in the art.

For administration, compounds of the present invention can be administered at a rate determined by factors that can include, but are not limited to, the LD₅₀ of the compound, the pharmacokinetic profile of the compound, contraindicated drugs, and the side effects of the compound at various concentrations, as applied to the mass and overall health of the subject. Administration can be accomplished via single or divided doses.

VIII. Methods for Treating or Preventing MKK7γ1-Mediated Disorders and Conditions

The compounds of the present invention and pharmaceutical compositions comprising a compound of the present invention can be administered to a subject suffering from a MKK7γ1-mediated disorder or condition. MKK7γ1-mediated disorders and conditions can be treated prophylactically, acutely, and chronically using compounds of the present invention, depending on the nature of the disorder or condition. Typically, the host or subject in each of these methods is human, although other mammals can also benefit from the administration of a compound of the present invention.

In therapeutic applications, the compounds of the present invention can be prepared and administered in a wide variety of oral and parenteral dosage forms. Thus, the compounds of the present invention can be administered by injection, that is, intravenously, intramuscularly, intracutaneously, subcutaneously, intraduodenally, or intraperitoneally. Also, the compounds described herein can be administered by inhalation, for example, intranasally. Additionally, the compounds of the present invention can be administered transdermally. In certain embodiments, the compounds of the present invention are delivered orally.

The compounds utilized in the pharmaceutical method of the invention can be administered at the initial dosage of about 0.001 mg/kg to about 100 mg/kg daily. In certain embodiments, the daily dose range is from about 0.1 mg/kg to about 10 mg/kg. The dosages, however, may be varied depending upon the requirements of the subject, the severity of the condition being treated, and the compound being employed. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. For convenience, the total daily dosage may be divided and administered in portions during the day, if desired.

In addition, the compounds of the present invention may be administered in combination with another suitable therapeutic agent. The suitable therapeutic agent that is administered to a subject suffering from a MKK7γ1-mediated disorder will depend on the nature and severity of the disorder. For example, suitable chemotherapeutic agents (e.g., TAXOL®, GLEEVEC® (Imatinib Mesylate), adriamycin, daunomycin, cisplatin, etoposide, a vinca alkaloid, vinblastine, vincristine, methotrexate, etc.) or radiation treatments can be administered in combination with a MKK7γ1 inhibitory compound to a subject suffering from cancer.

Furthermore, anti-inflammatory agents such as nonsteroidal anti-inflammatory agents (NSAIDS), piroxicam, diclofenac, naproxen, flurbiprofen, fenoprofen, ketoprofen ibuprofen, fenamates, mefenamic acid, indomethacin, sulindac, apazone, pyrazolones, phenylbutazone, aspirin, COX-2 inhibitors, CELEBREX® (celecoxib), VIOXX® (rofecoxib), and BEXTRA® (valdecoxib) may be administered in combination with a MKK7γ1 inhibitory to a subjects suffering from an inflammatory disease (e.g., rheumatoid arthritis, osteoarthritis, autoimmune disease, etc.).

Typically, the host or subject in each of these methods is human, although other animals can also benefit from the foregoing treatments.

IX. Methods of Detecting MKK7γ1 Sequences

The present invention also provides methods for detection of MKK7γ1 nucleic acid sequences. In addition, kits are provided that contain MKK7γ1 specific reagents that can be used to detect MKK7γ1 nucleic acids, such as specific probes and primers. The methods, kits, and the assays described herein can be used for the identification of cells or samples that contain MKK7γ1 sequences (e.g., MKK7γ1 mRNA). Biological samples can be tested for the presence of a MKK7γ1 nucleic acid using techniques that are known in the art. Biological samples are samples of tissue, fluid, blood, saliva, stool, urine, biopsy material, tumor cells, tumor biopsy, etc. that can be taken from a subject.

Nucleic acid assays for the presence of MKK7γ1 DNA and RNA in a sample include numerous techniques are known to those skilled in the art. Techniques such as Southern analysis, Northern analysis, dot blots, RNase protection, high density oligonucleotide arrays, S1 analysis, amplification techniques such as PCR and LCR, and in situ hybridization can be used as assays. For example a Northern blot of RNA from a biological sample can be probed with a labeled MKK7γ1 nucleic acid sequence such as a labeled oligonucleotide comprising at least 15 contiguous nucleotides of SEQ ID NO: 16. Labels include radiolabels such as ³²P, ³¹P, ³H, ³⁵S, and ¹⁴C as well as fluorescent labels. The presence of hybridized probe to a MKK7γ1 sequence can be visualized by autoradiography.

Alternatively, PCR using MKK7γ1 primers can be carried out on cDNA that has been reverse transcribed from an RNA sample. Examples of PCR primers that can be used to detect MKK7γ1 in a biological sample, e.g., a tumor or cancer biopsy, include, but are not limited SEQ ID NO: 15 and SEQ ID NO: 16. Those of skill in the art will recognize a wide variety of primers that can be used to detect MKK7γ1 nucleic acid sequences. The PCR can be carried out on MKK7γ1 cDNA sequences that have been reverse transcribed from a sample containing MKK7γ1 RNA using a reverse transcriptase, a primer, and suitable reaction components (see e.g., Innis et al. eds., PCR protocols: A guide to methods and applications. Academic Press, Inc., 1990).

The present invention also provides for kits for detecting nucleic acids. Such kits can be comprised of a container, MKK7γ1 primers or nucleic acids (e.g., SEQ ID NO: 16) and instructions for their use.

### EXAMPLES

It is understood that the examples and embodiments described herein are for illustrative purposes only and that various modifications or changes in light thereof will be suggested to persons skilled in the art and are to be included within the spirit and purview of this application and scope of the appended claims.

### Example 1

Isolation of a MKK7γ1 Nucleic Acid

A 5′ primer (SEQ ID NO: 18) and a 3′ primer (SEQ ID NO: 19) were used to amplify sequences from human skeletal muscle cDNA using the PCR.

A 50 μl reaction was prepared with 43 μL of PCR SuperMix (Invitrogen Corp., Carlsbad, Calif.), 1 μL of Human Skeletal Muscle QUICK-Clone™ cDNA (Clontech Laboratories, Inc., Palo Alto, Calif.), 2.5 μL of SEQ ID NO: 13, 2.5 μL of SEQ ID NO: 14, 2.5 μL of DMSO, and 1 μL of HiFi Taq Polymerase (Invitrogen Corp.).

The reaction was incubated in a thermocycler in four successive stages. In the first stage, the reaction was held at 95° C. for 3 minutes. In the second stage, the reaction was subjected to 35 cycles of 95° C. for 1 minute, 64° C. for 1 minute, 72° C. for 2 minutes. In the third stage, the reaction was held at 72° C. for 10 minutes. In the fourth stage the reaction was held at 4° C. until the sample was removed from the thermocycler. The samples were electrophoresed on a 1.5% (w/v) agarose gel and stained with ethidium bromide. Bands were excised from the gel, subcloned, and sequenced.

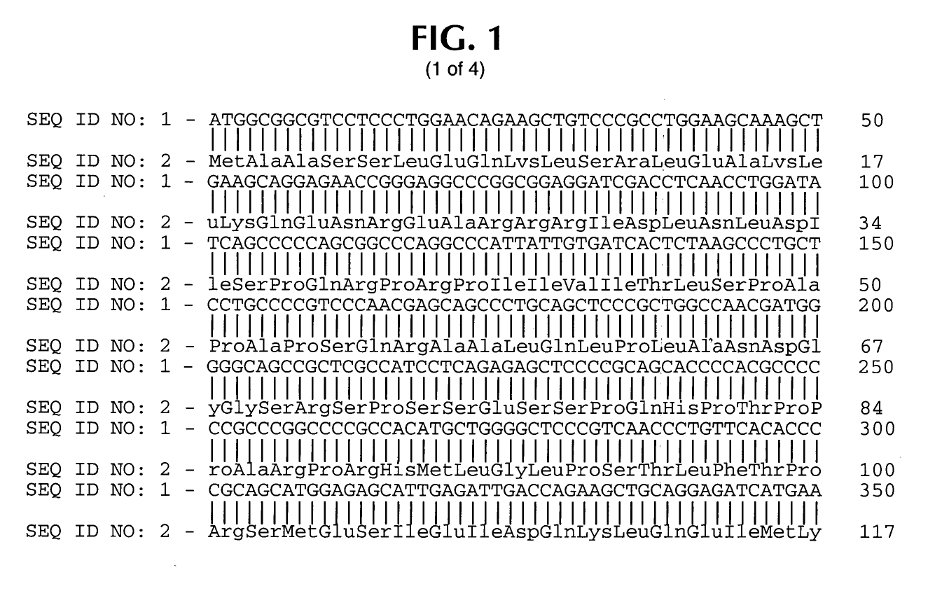

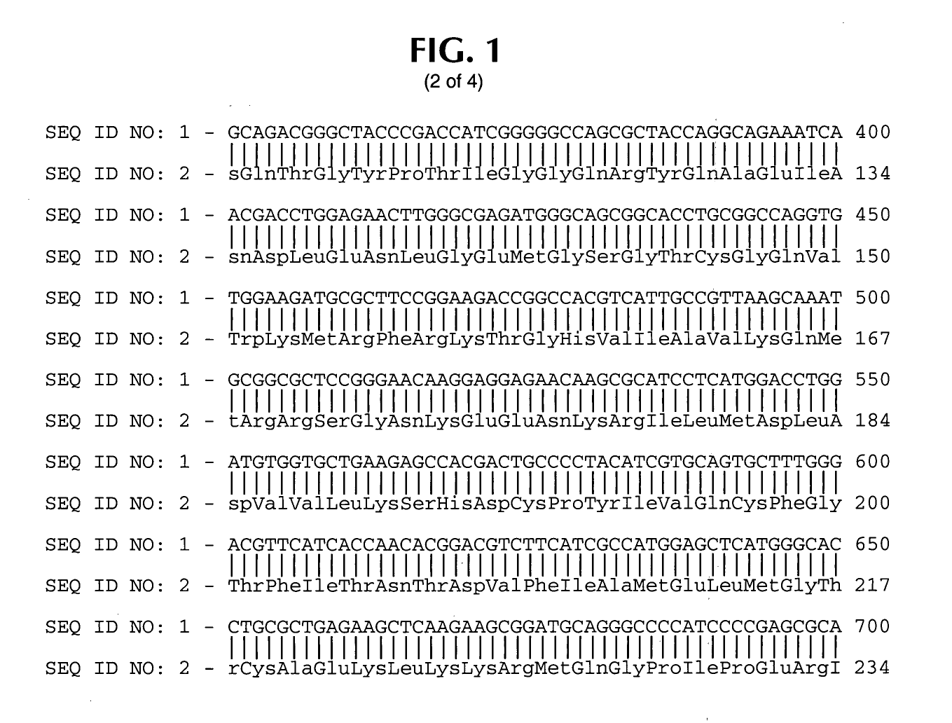

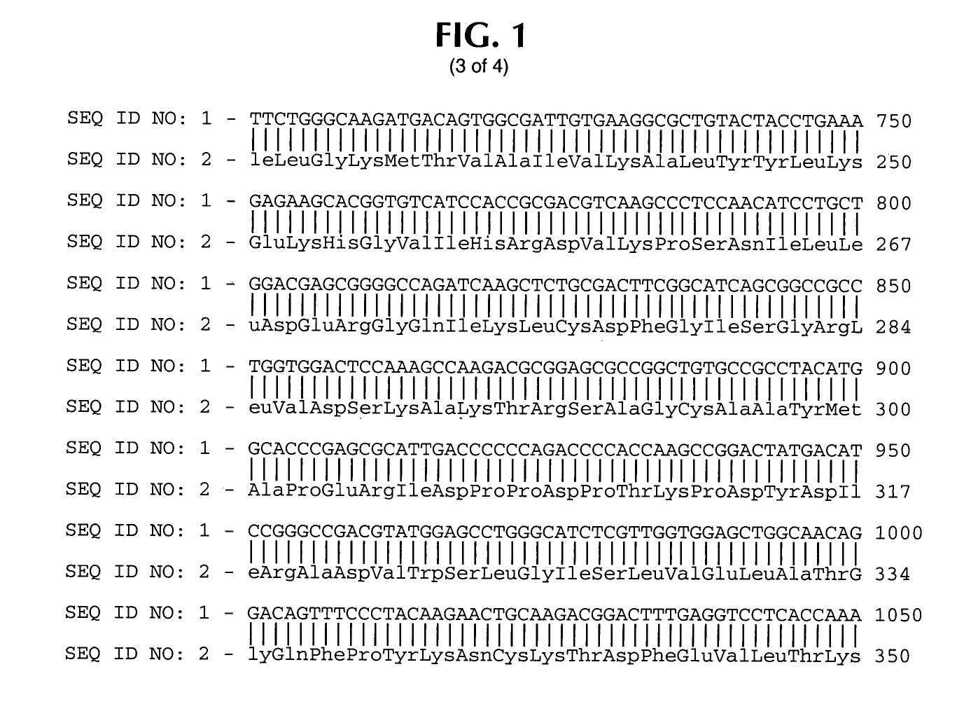

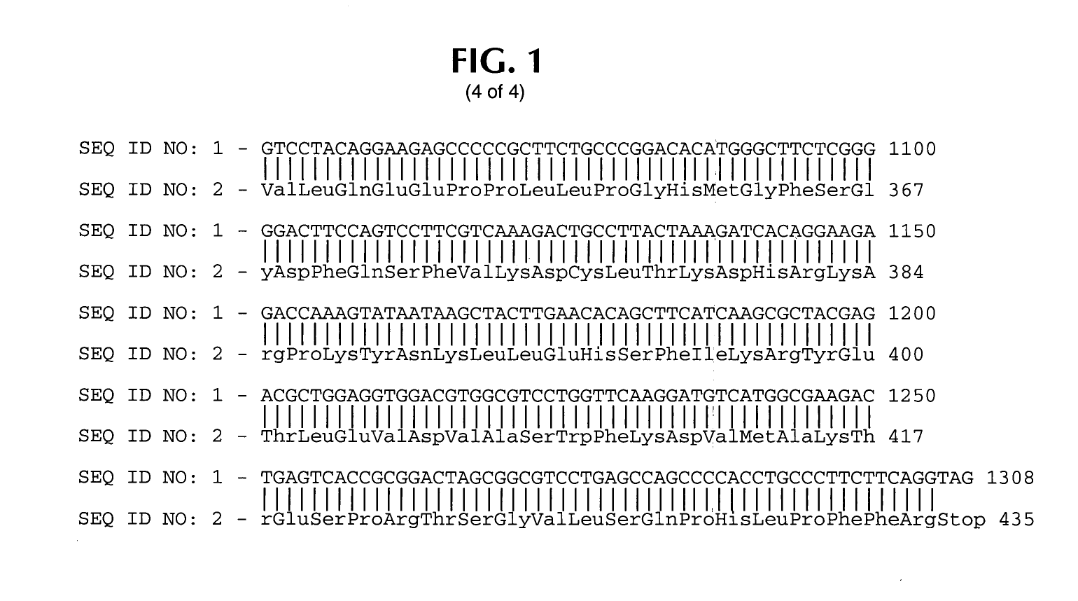

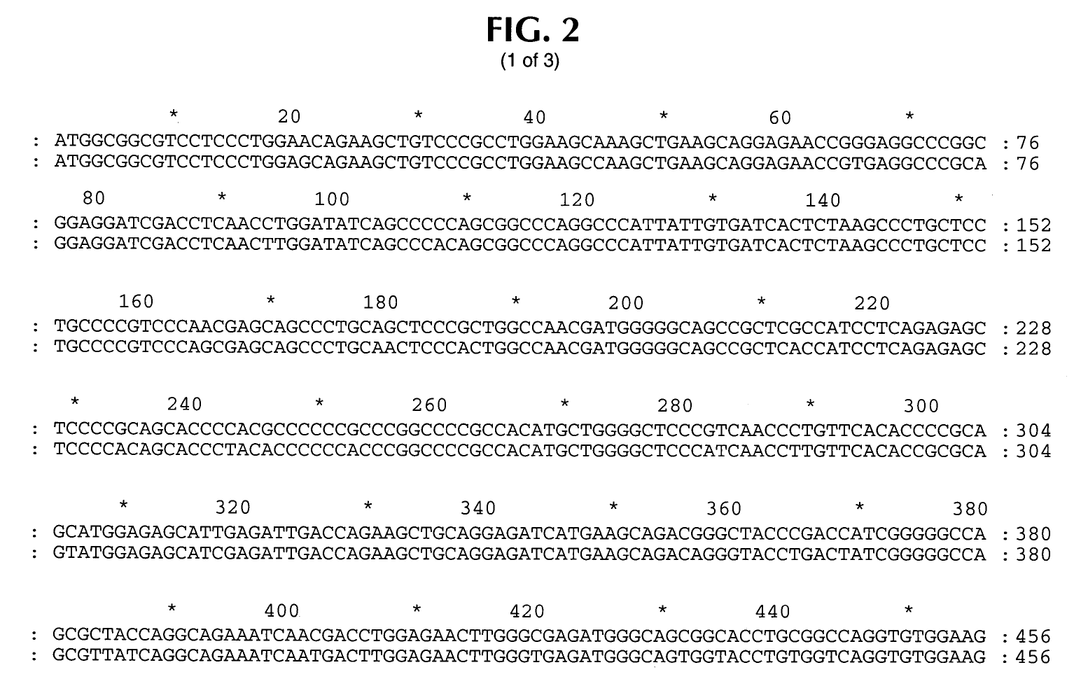

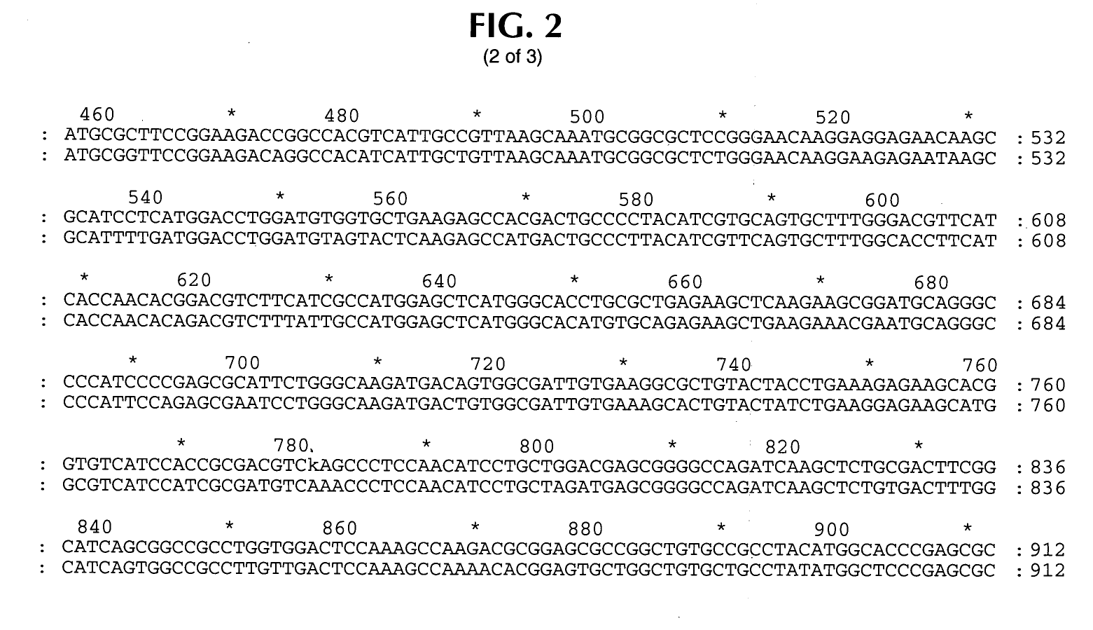

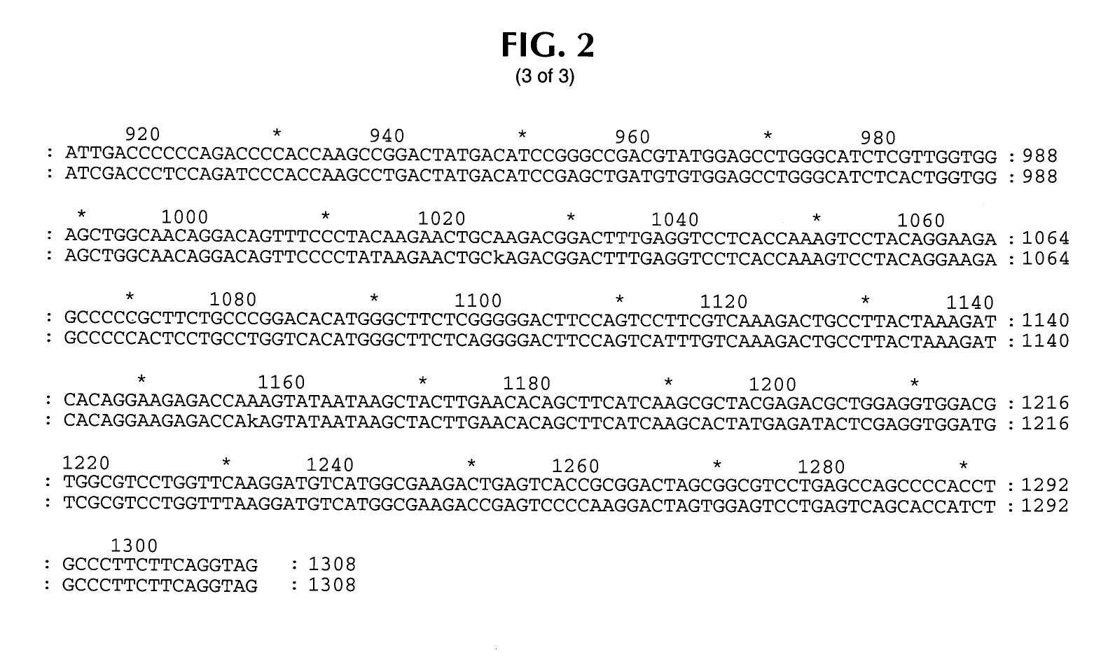

A sequence was identified (SEQ ID NO: 1; top line of FIG. 1) that encodes a polypeptide of SEQ ID NO: 2 (bottom line of FIG. 1). An alignment of the nucleotide (SEQ ID NO: 1) sequence to a mouse MKK7γ1 nucleotide sequence (SEQ ID NO: 11) (Tournier et al., supra., 1999) is depicted in FIG. 2. The alignment was performed using the GeneDoc Program, Version 2.6.001, which is distributed over the Internet by Karl Nicholas of the Pittsburgh Supercomputing Center, Pittsburgh, Pa. (Nicholas et al. GeneDoc: Analysis and Visualization of Genetic Variation. EMBNEW.NEWS, 1997;4:14). The alignment indicates that SEQ ID NO: 1 and SEQ ID NO: 11 have 90.5% nucleotide identity to each other over 1308 nucleotides.

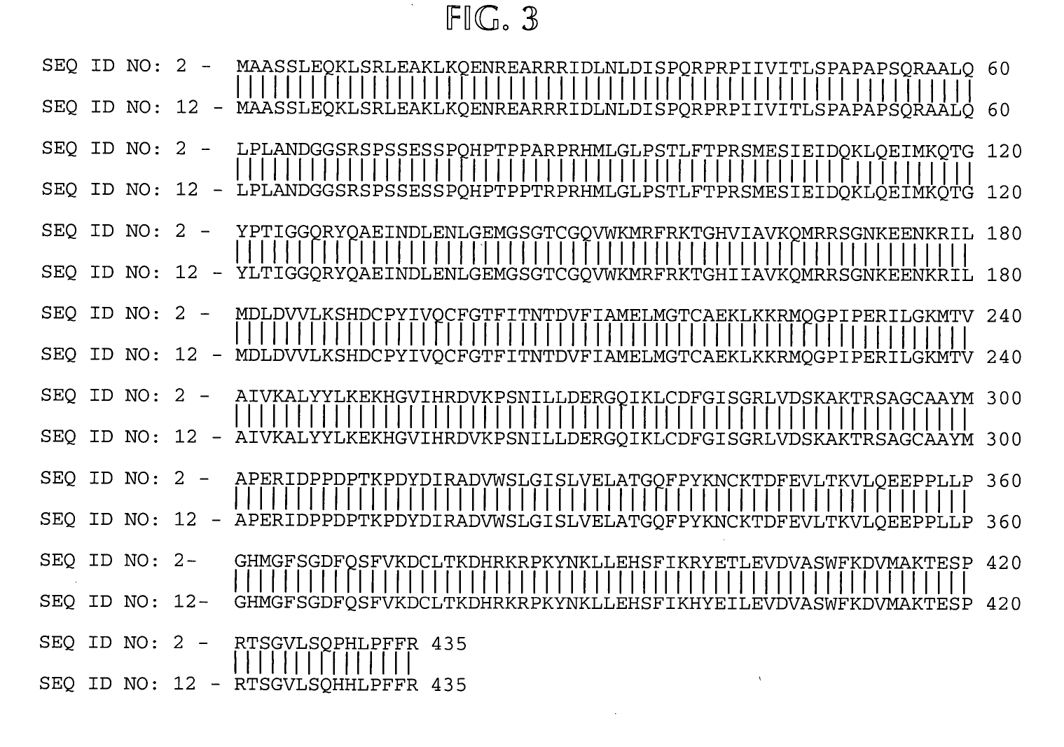

An alignment of the amino acid sequence (SEQ ID NO: 2) to a mouse MKK7γ1 amino acid sequence (SEQ ID NO: 12) (Tournier et al., supra., 1999) is depicted in FIG. 3. The alignment was performed using the GeneDoc Program Version 2.6.001. The alignment indicates that SEQ ID NO: 2 and SEQ ID NO: 12 have 98.6% amino acid identity (429/435 amino residues) to each other over 435 amino acids.

### Example 2

PCR Analysis of MKK7γ1 mRNA Expression

PCR amplification of Clontech MTC™ cDNA panels was used to assess expression profiles of SEQ ID NO: 1. The 5′ primer (SEQ ID NO: 13; ATGGCGGCGTCCTCCCTGGAACAGAAG) used in the PCR corresponds to the nucleotides 1 to 27 of SEQ ID NO: 1. The 3′ primer (SEQ ID NO: 14; GGCAGGAGCAGGGCTTAGAGTGATCACAATAATGG) used in the PCR is not complementary to a human MKK7° C. sequence (SEQ ID NO: 20) but is complementary to SEQ ID NO: 1. Thus, this primer set is designed to detect human MKK7γ1 (SEQ ID NO: 1) sequences and not a human MKK7α sequence (e.g., SEQ ID NO: 20). Amplification of SEQ ID NO: 1 with those primers is expected to result in a 156 bp PCR amplification product (SEQ ID NO: 15).

Panels of first strand cDNA samples were used as templates (Clontech Laboratories). Clontech Multiple Tissue cDNA (MTC™) panels are made from poly A+ RNA that is normalized to mRNA levels of four housekeeping genes.

The cDNAs were from the Clontech Human MTC™ Panel I (heart, brain, placenta, lung, liver, skeletal muscle, kidney, and pancreas); the Clontech Human Immune System MTC™ Panel (bone marrow, fetal liver, peripheral blood leukocyte, lymph node, spleen, thymus, and tonsil); and the Clontech Human Tumor MTC™ Panel (Colon Adenocarcinoma (CX-1), pancreatic carcinoma, prostatic adenocarcinoma, colon adenocarcinoma (GI-112), breast carcinoma, ovarian carcinoma, lung carcinoma (LX-1), lung carcinoma (GI-117)) (Clontech Laboratories).

Amplification Reaction

A series of 50 μL reactions were set up containing 36 μL of distilled water, 2 μL of a solution containing 10 μM of each primer, 5 μL of 10×TITANIUM™ Taq PCR Buffer (Clontech Laboratories, Inc.) (400 mM Tricine-KOH (pH 8.0), 160 mM KCl, 35 mM MgCl₂, 37.5 μg/mL Bovine Serum Albumin), 1 μL of 50×dNTP Mix (10 mM each of dATP, dCTP, dGTP, and dTTP), 1 μL of TITANIUM® Taq DNA Polymerase (Clontech Laboratories, Inc.) and 5 μL of the respective cDNA being amplified.

The reactions were incubated in a thermocycler in four successive stages. In the first stage, the reactions were held at 94° C. for 1 minute. In the second stage, the reactions were subjected to 38 cycles of 94° C. for 30 seconds, 63° C. for 1 minute, 72° C. for 1 minute. In the third stage, the reactions were held at 72° C. for 10 minutes. In the fourth stage the reactions were held at 4° C. until the sample was removed from the thermocycler of the respective cDNA being amplified.

Results

The reactions were electrophoresed on a 1.5% (w/v) agarose gel. The gel was then stained with ethidium bromide and a photograph was taken of the gel on an ultra-violet light box. In reactions that amplified a 156 bp band, the identity of the band as SEQ ID NO: 15 was confirmed by DNA sequencing. In the human tissue panel, the strongest expression of SEQ ID NO: 15 was exhibited in kidney (data not shown). Weaker expression of SEQ ID NO: 15 was seen in heart, brain, placenta, lung, liver, skeletal muscle, and pancreas (data not shown).

From the human immune panel, the strongest expression of SEQ ID NO: 15 was seen in lymph node (data not shown). Weaker expression of SEQ ID NO: 15 was seen in bone marrow, fetal liver, peripheral blood lymphocyte, and spleen. The lowest expression was in tonsil (data not shown). The thymus sample showed no visible expression of (data not shown).

In the reactions corresponding to the Clontech Human Tumor Panel, the strongest expression of SEQ ID NO: 15 was seen in breast carcinoma and lung (LX-1) carcinoma, followed by colon adenocarcinoma (CX-1) and colon adenocarcinoma (GI-112) (data not shown). The lowest amount of expression of SEQ ID NO: 15 was seen in pancreatic carcinoma, prostatic adenocarcinoma, ovarian carcinoma, and lung carcinoma (GI-117) (data not shown).

### Example 3

Northern Blot Analysis

Northern blot analysis of Clontech Multiple Tissue Northern (MTN®) 12 lane human blot was used to analyze mRNA expression profiles for MKK7γ1. The Clontech MTN® 12 lane human blot contains electrophoresed and blotted mRNAs isolated from human brain, colon, heart, kidney, liver, lung, peripheral blood leukocytes, placenta, skeletal muscle, small intestine, spleen, and thymus. Ambion's KinaseMax 5′ end labeling kit (Ambion, Inc., Austin, Tex.) was used to radiolabel a MKK7γ1 sequence (SEQ ID NO: 16) with ³²P. A nylon membrane of a Clontech MTN® (Catalog No. 7780-1) was first prehybridized with Clontech ExpressHyb™ Hybridization Solution at 37° C. The radiolabeled SEQ ID NO: 16 sequence was then hybridized to the 12 lane blot in the Clontech ExpressHyb™ Hybridization Solution at 37° C. The blot was rinsed several times at room temperature (RT) in 2×SSC, 0.1% SDS, once for 10 minutes in 0.5×SSC, 0.1% SDS at room temperature, once for 15 minutes in 0.5×SSC, 0.1% SDS at room temperature, and then once at 55° C. with 0.5×SSC, 0.1% SDS for 15 minutes. Band corresponding to MKK7γ1 sequences were visualized by autoradiography of the blot. Bands of the appropriate size were seen in skeletal muscle and placenta.

### Example 4

Northern Blot Analysis

SEQ ID NO: 16 was radiolabeled with ³²P as in Example 3. A nylon membrane of a Clontech Human Immune Tissue MTN® blot (Clontech Laboratories, Catalog No. 7780-1), containing blotted mRNAs isolated from human spleen, lymph node, thymus, peripheral blood leukocytes, bone marrow and fetal liver was used in this Example. The blot was first prehybridized with 10 mL of prehybridization solution at 73° C. for 2 hours. The prehybridization solution was made up of 3 mL of 20×SSC, 1 mL of 50× Denhardt's solution, 0.1 mL of salmon sperm DNA (10 mg/mL), 0.5 mL of 10% SDS, and 5.4 mL of distilled water. The immune MTN blot was then hybridized with 10 mL of hybridization solution containing the radiolabeled SEQ ID NO: 16 overnight at 73° C. The hybridization solution was made up of 3 mL of 20×SSC, 1 mL of 50× Denhardt's solution, 0.1 mL of salmon sperm DNA (10 mg/mL), 0.5 mL of 10% SDS, 0.2 mL of EDTA (pH 8.0), and 5.2 mL of distilled water.

The blot was washed twice at room temperature in 2×SSC, 0.1% SDS for 5 minutes each time. The blot was then washed twice for 5 minutes each in 0.5×SSC, 0.1% SDS at room temperature, once for 15 minutes in 0.5×SSC, 0.1% SDS at 50° C., once for 15 minutes in 0.5×SSC, 0.1% SDS at 55° C., and then 3 times for 15 minutes each time in 0.5×SSC, 0.1% SDS at 65° C. Band corresponding to MKK7γ1 sequences were visualized by autoradiography of the blot. A band of the correct transcript size was seen in fetal liver.

### Example 5

Expression and Purification of Histidine Tagged-MKK7γ1

A MKK7γ1 nucleic acid (SEQ ID NO: 1) was subcloned into a pET28b bacterial expression vector (Novagen, Inc., Madison, Wis.). The pET28b vector permits the expression of a histidine-tagged MKK7γ1 in Escherichia coli. The plasmid was transformed into either BL21(DE3) cells (Novagen, Inc.) or BL21(DE3)-RP cells (Stratagene Inc., La Jolla, Calif.). Typically, the cells from a freshly transformed plate of bacteria were grown in 2×YT media at 37° to an OD₆₀₀ of 0.500 and then induced with 500 μM IPTG for 2.5 hours at 30° C.

Expression was confirmed by western blotting using a 6×His monoclonal antibody (Clontech Laboratories). In shakeflask expression studies using IL volumes, the histidine-tagged-MKK7γ1 protein was readily visualized on a SDS-PAGE gel stained with GelCode Blue (Pierce Biotechnology, Inc., Rockford, Ill.).

Cell pastes were resuspended in cell lysis buffer A (50 mM Hepes, pH 7.5/300 mM NaCl/2 mM β-Me/10 mM imidazole). MgCl₂ was added to a final concentration of 10 mM. Then, 5 μL benzonase was added, and the cells were lysed through the use of a microfluidizer. The cell suspensions were centrifuged at 11,000 rpm for 40 minutes to separate the soluble fraction from the pellet. Lysate was applied at 5 mL/minute to a 5 mL HiTrap Chelating HP column (Amersham Biosciences Corp., Piscataway, N.J.), previously equilibrated in buffer A. The column was eluted with a linear gradient of 10 to 300 mM imidazole collecting 2 mL fractions.

### Example 6

Protein Kinase Assays

Two-step cascade kinase assays were performed in 100 μL total volume (50 mM Tris, pH 7.4, 10 mM MgCl₂, 1 mM DTT, 50 μM ATP and 5 μCi γ-³²P-ATP per reaction). Active MEKK1 (Upstate, Inc., Lake Placid, N.Y.) (0.5 μg/reaction) was preincubated with either 1 μg of His-tagged MKK7γ1 or 1 μg unactive MKK7β1 (UBI) for 40 minutes at 30° C. The recombinant MKK7β was used as a positive control for activation by MEKK1 and for the phoshorylation of JNK1. Either 1 μg unactive JNK1 (Upstate, Inc., Lake Placid, N.Y.) or 1 μg unactive p38 was then added to the reaction as substrate for MKK7γ1. These reactions were stopped after 45 more minutes with the addition of Laemmli sample buffer. Fractions of the kinase reactions were subjected to SDS/PAGE. The gels were stained using GelCode Blue followed by destaining with water. The gels were dried and exposed to X-ray film. Bands were visualized by autoradiography.

The His-tagged MKK7γ1 protein and the MKK7β1 protein was phosphorylated by MEKK1 and in turn was able to phosphorylate JNK1 (date not shown). MKK7γ1 was unable to phosphorylate p38 (data not shown). These results indicate that SEQ ID NO: 1 encodes a protein kinase capable of phosphorylating JNK1.

All publications, patents, and patent applications cited herein are hereby incorporated by reference in their entirety for all purposes.

What is claimed is:

1. An isolated nucleic acid comprising:
a nucleotide sequence encoding the 435 amino acid polypeptide of SEQ ID NO: 17,
wherein X²⁸⁷ is Asp, Glu, or Ser;
wherein X²⁹¹ is Asp, Glu, or Thr; and
wherein X²⁹³ is Asp, Glu, or Ser.

2. The nucleic acid of claim 1, wherein said isolated nucleic acid encodes a polypeptide selected from the group consisting of:
SEQ ID NO: 2, SEQ ID NO: 4, SEQ ID NO: 6, SEQ ID NO: 8, SEQ ID NO: 10, and SEQ ID NO: 23.

3. The nucleic acid of claim 1, wherein said nucleic comprises a nucleic acid selected from the group consisting of:
SEQ ID NO: 1, SEQ ID NO: 3, SEQ ID NO: 5, SEQ ID NO: 7, SEQ ID NO: 9, and SEQ ID NO: 22.

4. The nucleic acid of claim 3, wherein said isolated nucleic acid consists of a nucleotide acid selected from the group consisting of:
SEQ ID NO: 1, SEQ ID NO: 3, SEQ ID NO: 5, SEQ ID NO: 7, SEQ ID NO: 9, and SEQ ID NO: 22.

5. An isolated polypeptide comprising:
the 435 amino acid polypeptide of SEQ ID NO: 17:
wherein X²⁸⁷ is Asp, Glu, or Ser;
wherein X²⁹¹ is Asp, Glu, or Thr; and
wherein X²⁹³ is Asp, Glu, or Ser.

6. The polypeptide of claim 5, wherein said polypeptide comprises a polypeptide selected from the group consisting of:
SEQ ID NO: 2, SEQ ID NO:4, SEQ ID NO: 6, SEQ ID NO: 8, SEQ ID NO: 10, and SEQ ID NO:23.

7. The polypeptide of claim 6, wherein said polypeptide consists of a polypeptide selected from the group consisting of:
SEQ ID NO: 2, SEQ ID NO:4, SEQ ID NO: 6, SEQ ID NO: 8, SEQ ID NO: 10, and SEQ ID NO: 23.

8. An expression cassette comprising:
a promoter operably linked to a nucleic acid encoding the 435 amino acid polypeptide of SEQ ID NO: 17,
wherein X²⁸⁷ is Asp, Glu, or Ser;
wherein X²⁹¹ is Asp, Glu, or Thr; and
wherein X²⁹³ is Asp, Glu, or Ser.

9. The cassette of claim 8, wherein said nucleic acid encodes a polypeptide selected from the group consisting of:
SEQ ID NO: 2, SEQ ID NO: 4, SEQ ID NO: 6, SEQ ID NO: 8, SEQ ID NO: 10, and SEQ ID NO: 23.

10. The cassette of claim 8, wherein said nucleic comprises a nucleic acid selected from the group consisting of:
SEQ ID NO: 1, SEQ ID NO: 3, SEQ ID NO: 5, SEQ ID NO: 7, SEQ ID NO: 9, and SEQ ID NO: 22.

11. A method for assaying protein kinase activity comprising:
incubating an isolated polypeptide comprising:
the 435 amino acid polypeptide of SEQ ID NO: 17,
wherein X²⁸⁷ is Asp, Glu, or Ser;
wherein X²⁹¹ is Asp, Glu, or Thr; and
wherein X²⁹³ is Asp, Glu, or Ser;
in the presence of ATP and a MKK7γ1 substrate; and
determining whether said MKK7γ1 substrate is phosphorylated.

12. The method of claim 11, wherein said ATP is γ-³²P-ATP.

13. The method of claim 12, wherein said isolated polypeptide comprises SEQ ID NO: 2, SEQ ID NO: 4, SEQ ID NO: 6, SEQ ID NO: 8, SEQ ID NO: 10, or SEQ ID NO: 23.

14. The method of claim 11, wherein said method further comprises incubating said isolated polypeptide in the presence of a candidate therapeutic agent having a molecular weight of between 100 Da and 1000 Da.

15. The method of claim 11, wherein said MKK7γ1 substrate is JNK1.


SEQUENCE LISTING (23 sequences)

SEQ ID NO: 1 - DNA, 1308 bp, Human
atggcggcgtcctccctggaacagaagctgtcccgcctggaagcaaagctgaagcaggagaaccgggaggcccggcggaggatcgacctcaacctggatatcagcccccagcggcccaggcccattattgtgatcactctaagccctgctcctgccccgtcccaacgagcagccctgcagctcccgctggccaacgatgggggcagccgctcgccatcctcagagagctccccgcagcaccccacgccccccgcccggccccgccacatgctggggctcccgtcaaccctgttcacaccccgcagcatggagagcattgagattgaccagaagctgcaggagatcatgaagcagacgggctacccgaccatcgggggccagcgctaccaggcagaaatcaacgacctggagaacttgggcgagatgggcagcggcacctgcggccaggtgtggaagatgcgcttccggaagaccggccacgtcattgccgttaagcaaatgcggcgctccgggaacaaggaggagaacaagcgcatcctcatggacctggatgtggtgctgaagagccacgactgcccctacatcgtgcagtgctttgggacgttcatcaccaacacggacgtcttcatcgccatggagctcatgggcacctgcgctgagaagctcaagaagcggatgcagggccccatccccgagcgcattctgggcaagatgacagtggcgattgtgaaggcgctgtactacctgaaagagaagcacggtgtcatccaccgcgacgtcaagccctccaacatcctgctggacgagcggggccagatcaagctctgcgacttcggcatcagcggccgcctggtggactccaaagccaagacgcggagcgccggctgtgccgcctacatggcacccgagcgcattgaccccccagaccccaccaagccggactatgacatccgggccgacgtatggagcctgggcatctcgttggtggagctggcaacaggacagtttccctacaagaactgcaagacggactttgaggtcctcaccaaagtcctacaggaagagcccccgcttctgcccggacacatgggcttctcgggggacttccagtccttcgtcaaagactgccttactaaagatcacaggaagagaccaaagtataataagctacttgaacacagcttcatcaagcgctacgagacgctggaggtggacgtggcgtcctggttcaaggatgtcatggcgaagactgagtcaccgcggactagcggcgtcctgagccagccccacctgcccttcttcaggtag

SEQ ID NO: 2 - protein, 435 aa, Human [sequence omitted]

SEQ ID NO: 3 - DNA, 1308 bp, Human
atggcggcgtcctccctggaacagaagctgtcccgcctggaagcaaagctgaagcaggagaaccgggaggcccggcggaggatcgacctcaacctggatatcagcccccagcggcccaggcccattattgtgatcactctaagccctgctcctgccccgtcccaacgagcagccctgcagctcccgctggccaacgatgggggcagccgctcgccatcctcagagagctccccgcagcaccccacgccccccgcccggccccgccacatgctggggctcccgtcaaccctgttcacaccccgcagcatggagagcattgagattgaccagaagctgcaggagatcatgaagcagacgggctacccgaccatcgggggccagcgctaccaggcagaaatcaacgacctggagaacttgggcgagatgggcagcggcacctgcggccaggtgtggaagatgcgcttccggaagaccggccacgtcattgccgttaagcaaatgcggcgctccgggaacaaggaggagaacaagcgcatcctcatggacctggatgtggtgctgaagagccacgactgcccctacatcgtgcagtgctttgggacgttcatcaccaacacggacgtcttcatcgccatggagctcatgggcacctgcgctgagaagctcaagaagcggatgcagggccccatccccgagcgcattctgggcaagatgacagtggcgattgtgaaggcgctgtactacctgaaagagaagcacggtgtcatccaccgcgacgtcaagccctccaacatcctgctggacgagcggggccagatcaagctctgcgacttcggcatcagcggccgcctggtggacgacaaagccaaggatcggagcgccggctgtgccgcctacatggcacccgagcgcattgaccccccagaccccaccaagccggactatgacatccgggccgacgtatggagcctgggcatctcgttggtggagctggcaacaggacagtttccctacaagaactgcaagacggactttgaggtcctcaccaaagtcctacaggaagagcccccgcttctgcccggacacatgggcttctcgggggacttccagtccttcgtcaaagactgccttactaaagatcacaggaagagaccaaagtataataagctacttgaacacagcttcatcaagcgctacgagacgctggaggtggacgtggcgtcctggttcaaggatgtcatggcgaagactgagtcaccgcggactagcggcgtcctgagccagccccacctgcccttcttcaggtag

SEQ ID NO: 4 - protein, 435 aa, Human
MAASSLEQKLSRLEAKLKQENREARRRIDLNLDISPQRPRPIIVITLSPAPAPSQRAALQLPLANDGGSRSPSSESSPQHPTPPARPRHMLGLPSTLFTPRSMESIEIDQKLQEIMKQTGYPTIGGQRYQAEINDLENLGEMGSGTCGQVWKMRFRKTGHVIAVKQMRRSGNKEENKRILMDLDVVLKSHDCPYIVQCFGTFITNTDVFIAMELMGTCAEKLKKRMQGPIPERILGKMTVAIVKALYYLKEKHGVIHRDVKPSNILLDERGQIKLCDFGISGRLVDDKAKDRSAGCAAYMAPERIDPPDPTKPDYDIRADVWSLGISLVELATGQFPYKNCKTDFEVLTKVLQEEPPLLPGHMGFSGDFQSFVKDCLTKDHRKRPKYNKLLEHSFIKRYETLEVDVASWFKDVMAKTESPRTSGVLSQPHLPFFR

SEQ ID NO: 5 - DNA, 1308 bp, Human
atggcggcgtcctccctggaacagaagctgtcccgcctggaagcaaagctgaagcaggagaaccgggaggcccggcggaggatcgacctcaacctggatatcagcccccagcggcccaggcccattattgtgatcactctaagccctgctcctgccccgtcccaacgagcagccctgcagctcccgctggccaacgatgggggcagccgctcgccatcctcagagagctccccgcagcaccccacgccccccgcccggccccgccacatgctggggctcccgtcaaccctgttcacaccccgcagcatggagagcattgagattgaccagaagctgcaggagatcatgaagcagacgggctacccgaccatcgggggccagcgctaccaggcagaaatcaacgacctggagaacttgggcgagatgggcagcggcacctgcggccaggtgtggaagatgcgcttccggaagaccggccacgtcattgccgttaagcaaatgcggcgctccgggaacaaggaggagaacaagcgcatcctcatggacctggatgtggtgctgaagagccacgactgcccctacatcgtgcagtgctttgggacgttcatcaccaacacggacgtcttcatcgccatggagctcatgggcacctgcgctgagaagctcaagaagcggatgcagggccccatccccgagcgcattctgggcaagatgacagtggcgattgtgaaggcgctgtactacctgaaagagaagcacggtgtcatccaccgcgacgtcaagccctccaacatcctgctggacgagcggggccagatcaagctctgcgacttcggcatcagcggccgcctggtggacgacaaagccaaggagcggagcgccggctgtgccgcctacatggcacccgagcgcattgaccccccagaccccaccaagccggactatgacatccgggccgacgtatggagcctgggcatctcgttggtggagctggcaacaggacagtttccctacaagaactgcaagacggactttgaggtcctcaccaaagtcctacaggaagagcccccgcttctgcccggacacatgggcttctcgggggacttccagtccttcgtcaaagactgccttactaaagatcacaggaagagaccaaagtataataagctacttgaacacagcttcatcaagcgctacgagacgctggaggtggacgtggcgtcctggttcaaggatgtcatggcgaagactgagtcaccgcggactagcggcgtcctgagccagccccacctgcccttcttcaggtag

SEQ ID NO: 6 - protein, 435 aa, Human
MAASSLEQKLSRLEAKLKQENREARRRIDLNLDISPQRPRPIIVITLSPAPAPSQRAALQLPLANDGGSRSPSSESSPQHPTPPARPRHMLGLPSTLFTPRSMESIEIDQKLQEIMKQTGYPTIGGQRYQAEINDLENLGEMGSGTCGQVWKMRFRKTGHVIAVKQMRRSGNKEENKRILMDLDVVLKSHDCPYIVQCFGTFITNTDVFIAMELMGTCAEKLKKRMQGPIPERILGKMTVAIVKALYYLKEKHGVIHRDVKPSNILLDERGQIKLCDFGISGRLVDDKAKERSAGCAAYMAPERIDPPDPTKPDYDIRADVWSLGISLVELATGQFPYKNCKTDFEVLTKVLQEEPPLLPGHMGFSGDFQSFVKDCLTKDHRKRPKYNKLLEHSFIKRYETLEVDVASWFKDVMAKTESPRTSGVLSQPHLPFFR

SEQ ID NO: 7 - DNA, 1308 bp, Human
atggcggcgtcctccctggaacagaagctgtcccgcctggaagcaaagctgaagcaggagaaccgggaggcccggcggaggatcgacctcaacctggatatcagcccccagcggcccaggcccattattgtgatcactctaagccctgctcctgccccgtcccaacgagcagccctgcagctcccgctggccaacgatgggggcagccgctcgccatcctcagagagctccccgcagcaccccacgccccccgcccggccccgccacatgctggggctcccgtcaaccctgttcacaccccgcagcatggagagcattgagattgaccagaagctgcaggagatcatgaagcagacgggctacccgaccatcgggggccagcgctaccaggcagaaatcaacgacctggagaacttgggcgagatgggcagcggcacctgcggccaggtgtggaagatgcgcttccggaagaccggccacgtcattgccgttaagcaaatgcggcgctccgggaacaaggaggagaacaagcgcatcctcatggacctggatgtggtgctgaagagccacgactgcccctacatcgtgcagtgctttgggacgttcatcaccaacacggacgtcttcatcgccatggagctcatgggcacctgcgctgagaagctcaagaagcggatgcagggccccatccccgagcgcattctgggcaagatgacagtggcgattgtgaaggcgctgtactacctgaaagagaagcacggtgtcatccaccgcgacgtcaagccctccaacatcctgctggacgagcggggccagatcaagctctgcgacttcggcatcagcggccgcctggtggacgagaaagccaaggaccggagcgccggctgtgccgcctacatggcacccgagcgcattgaccccccagaccccaccaagccggactatgacatccgggccgacgtatggagcctgggcatctcgttggtggagctggcaacaggacagtttccctacaagaactgcaagacggactttgaggtcctcaccaaagtcctacaggaagagcccccgcttctgcccggacacatgggcttctcgggggacttccagtccttcgtcaaagactgccttactaaagatcacaggaagagaccaaagtataataagctacttgaacacagcttcatcaagcgctacgagacgctggaggtggacgtggcgtcctggttcaaggatgtcatggcgaagactgagtcaccgcggactagcggcgtcctgagccagccccacctgcccttcttcaggtag

SEQ ID NO: 8 - protein, 435 aa, Human
MAASSLEQKLSRLEAKLKQENREARRRIDLNLDISPQRPRPIIVITLSPAPAPSQRAALQLPLANDGGSRSPSSESSPQHPTPPARPRHMLGLPSTLFTPRSMESIEIDQKLQEIMKQTGYPTIGGQRYQAEINDLENLGEMGSGTCGQVWKMRFRKTGHVIAVKQMRRSGNKEENKRILMDLDVVLKSHDCPYIVQCFGTFITNTDVFIAMELMGTCAEKLKKRMQGPIPERILGKMTVAIVKALYYLKEKHGVIHRDVKPSNILLDERGQIKLCDFGISGRLVDEKAKDRSAGCAAYMAPERIDPPDPTKPDYDIRADVWSLGISLVELATGQFPYKNCKTDFEVLTKVLQEEPPLLPGHMGFSGDFQSFVKDCLTKDHRKRPKYNKLLEHSFIKRYETLEVDVASWFKDVMAKTESPRTSGVLSQPHLPFFR

SEQ ID NO: 9 - DNA, 1308 bp, Human
atggcggcgtcctccctggaacagaagctgtcccgcctggaagcaaagctgaagcaggagaaccgggaggcccggcggaggatcgacctcaacctggatatcagcccccagcggcccaggcccattattgtgatcactctaagccctgctcctgccccgtcccaacgagcagccctgcagctcccgctggccaacgatgggggcagccgctcgccatcctcagagagctccccgcagcaccccacgccccccgcccggccccgccacatgctggggctcccgtcaaccctgttcacaccccgcagcatggagagcattgagattgaccagaagctgcaggagatcatgaagcagacgggctacccgaccatcgggggccagcgctaccaggcagaaatcaacgacctggagaacttgggcgagatgggcagcggcacctgcggccaggtgtggaagatgcgcttccggaagaccggccacgtcattgccgttaagcaaatgcggcgctccgggaacaaggaggagaacaagcgcatcctcatggacctggatgtggtgctgaagagccacgactgcccctacatcgtgcagtgctttgggacgttcatcaccaacacggacgtcttcatcgccatggagctcatgggcacctgcgctgagaagctcaagaagcggatgcagggccccatccccgagcgcattctgggcaagatgacagtggcgattgtgaaggcgctgtactacctgaaagagaagcacggtgtcatccaccgcgacgtcaagccctccaacatcctgctggacgagcggggccagatcaagctctgcgacttcggcatcagcggccgcctggtggacgagaaagccaaggagcggagcgccggctgtgccgcctacatggcacccgagcgcattgaccccccagaccccaccaagccggactatgacatccgggccgacgtatggagcctgggcatctcgttggtggagctggcaacaggacagtttccctacaagaactgcaagacggactttgaggtcctcaccaaagtcctacaggaagagcccccgcttctgcccggacacatgggcttctcgggggacttccagtccttcgtcaaagactgccttactaaagatcacaggaagagaccaaagtataataagctacttgaacacagcttcatcaagcgctacgagacgctggaggtggacgtggcgtcctggttcaaggatgtcatggcgaagactgagtcaccgcggactagcggcgtcctgagccagccccacctgcccttcttcaggtag

SEQ ID NO: 10 - protein, 435 aa, Human
MAASSLEQKLSRLEAKLKQENREARRRIDLNLDISPQRPRPIIVITLSPAPAPSQRAALQLPLANDGGSRSPSSESSPQHPTPPARPRHMLGLPSTLFTPRSMESIEIDQKLQEIMKQTGYPTIGGQRYQAEINDLENLGEMGSGTCGQVWKMRFRKTGHVIAVKQMRRSGNKEENKRILMDLDVVLKSHDCPYIVQCFGTFITNTDVFIAMELMGTCAEKLKKRMQGPIPERILGKMTVAIVKALYYLKEKHGVIHRDVKPSNILLDERGQIKLCDFGISGRLVDEKAKERSAGCAAYMAPERIDPPDPTKPDYDIRADVWSLGISLVELATGQFPYKNCKTDFEVLTKVLQEEPPLLPGHMGFSGDFQSFVKDCLTKDHRKRPKYNKLLEHSFIKRYETLEVDVASWFKDVMAKTESPRTSGVLSQPHLPFFR

SEQ ID NO: 11 - DNA, 1308 bp, Mus musculus
atggcggcgtcctccctggagcagaagctgtcccgcctggaagccaagctgaagcaggagaaccgtgaggcccgcaggaggatcgacctcaacttggatatcagcccacagcggcccaggcccattattgtgatcactctaagccctgctcctgccccgtcccagcgagcagccctgcaactcccactggccaacgatgggggcagccgctcaccatcctcagagagctccccacagcaccctacaccccccacccggccccgccacatgctggggctcccatcaaccttgttcacaccgcgcagtatggagagcatcgagattgaccagaagctgcaggagatcatgaagcagacagggtacctgactatcgggggccagcgttatcaggcagaaatcaatgacttggagaacttgggtgagatgggcagtggtacctgtggtcaggtgtggaagatgcggttccggaagacaggccacatcattgctgttaagcaaatgcggcgctctgggaacaaggaagagaataagcgcattttgatggacctggatgtagtactcaagagccatgactgcccttacatcgttcagtgctttggcaccttcatcaccaacacagacgtctttattgccatggagctcatgggcacatgtgcagagaagctgaagaaacgaatgcagggccccattccagagcgaatcctgggcaagatgactgtggcgattgtgaaagcactgtactatctgaaggagaagcatggcgtcatccatcgcgatgtcaaaccctccaacatcctgctagatgagcggggccagatcaagctctgtgactttggcatcagtggccgccttgttgactccaaagccaaaacacggagtgctggctgtgctgcctatatggctcccgagcgcatcgaccctccagatcccaccaagcctgactatgacatccgagctgatgtgtggagcctgggcatctcactggtggagctggcaacaggacagttcccctataagaactgcaagacggactttgaggtcctcaccaaagtcctacaggaagagcccccactcctgcctggtcacatgggcttctcaggggacttccagtcatttgtcaaagactgccttactaaagatcacaggaagagaccaaagtataataagctacttgaacacagcttcatcaagcactatgagatactcgaggtggatgtcgcgtcctggtttaaggatgtcatggcgaagaccgagtccccaaggactagtggagtcctgagtcagcaccatctgcccttcttcaggtag

SEQ ID NO: 12 - protein, 435 aa, Mus musculus
MAASSLEQKLSRLEAKLKQENREARRRIDLNLDISPQRPRPIIVITLSPAPAPSQRAALQLPLANDGGSRSPSSESSPQHPTPPTRPRHMLGLPSTLFTPRSMESIEIDQKLQEIMKQTGYLTIGGQRYQAEINDLENLGEMGSGTCGQVWKMRFRKTGHIIAVKQMRRSGNKEENKRILMDLDVVLKSHDCPYIVQCFGTFITNTDVFIAMELMGTCAEKLKKRMQGPIPERILGKMTVAIVKALYYLKEKHGVIHRDVKPSNILLDERGQIKLCDFGISGRLVDSKAKTRSAGCAAYMAPERIDPPDPTKPDYDIRADVWSLGISLVELATGQFPYKNCKTDFEVLTKVLQEEPPLLPGHMGFSGDFQSFVKDCLTKDHRKRPKYNKLLEHSFIKHYEILEVDVASWFKDVMAKTESPRTSGVLSQHHLPFFR

SEQ ID NO: 13 - DNA, 27 bp, Artificial Sequence
  primer
atggcggcgtcctccctggaacagaag

SEQ ID NO: 14 - DNA, 35 bp, Artificial Sequence
  primer
ggcaggagcagggcttagagtgatcacaataatgg

SEQ ID NO: 15 - DNA, 156 bp, Human
atggcggcgtcctccctggaacagaagctgtcccgcctggaagcaaagctgaagcaggagaaccgggaggcccggcggaggatcgacctcaacctggatatcagcccccagcggcccaggcccattattgtgatcactctaagccctgctcctgcc

SEQ ID NO: 16 - DNA, 48 bp, Human
attattgtgatcactctaagccctgctcctgccccgtcccaacgagca

SEQ ID NO: 17 - protein, 435 aa, Human
  MISC_FEATURE (291)..(291): Xaa is Aspartic Acid, Glutamic Acid, or Threonine
MAASSLEQKLSRLEAKLKQENREARRRIDLNLDISPQRPRPIIVITLSPAPAPSQRAALQLPLANDGGSRSPSSESSPQHPTPPARPRHMLGLPSTLFTPRSMESIEIDQKLQEIMKQTGYPTIGGQRYQAEINDLENLGEMGSGTCGQVWKMRFRKTGHVIAVKQMRRSGNKEENKRILMDLDVVLKSHDCPYIVQCFGTFITNTDVFIAMELMGTCAEKLKKRMQGPIPERILGKMTVAIVKALYYLKEKHGVIHRDVKPSNILLDERGQIKLCDFGISGRLVDXKAKXRXAGCAAYMAPERIDPPDPTKPDYDIRADVWSLGISLVELATGQFPYKNCKTDFEVLTKVLQEEPPLLPGHMGFSGDFQSFVKDCLTKDHRKRPKYNKLLEHSFIKRYETLEVDVASWFKDVMAKTESPRTSGVLSQPHLPFFR

SEQ ID NO: 18 - DNA, 24 bp, Artificial Sequence
  Primer
gggaaacatatggcggcgtcctcc

SEQ ID NO: 19 - DNA, 28 bp, Artificial Sequence
  Primer
ggatccctacctgaagaagggcaggtgg

SEQ ID NO: 20 - DNA, 1260 bp, Human
atggcggcgtcctccctggaacagaagctgtcccgcctggaagcaaagctgaagcaggagaaccgggaggcccggcggaggatcgacctcaacctggatatcagcccccagcggcccaggcccaccctgcagctcccgctggccaacgatgggggcagccgctcgccatcctcagagagctccccgcagcaccccacgccccccgcccggccccgccacatgctggggctcccgtcaaccctgttcacaccccgcagcatggagagcattgagattgaccagaagctgcaggagatcatgaagcagacgggctacctgaccatcgggggccagcgctaccaggcagaaatcaacgacctggagaacttgggcgagatgggcagcggcacctgcggccaggtgtggaagatgcgcttccggaagaccggccacgtcattgccgttaagcaaatgcggcgctccgggaacaaggaggagaacaagcgcatcctcatggacctggatgtggtgctgaagagccacgactgcccctacatcgtgcagtgctttgggacgttcatcaccaacacggacgtcttcatcgccatggagctcatgggcacctgcgctgagaagctcaagaagcggatgcagggccccatccccgagcgcattctgggcaagatgacagtggcgattgtgaaggcgctgtactacctgaaggagaagcacggtgtcatccaccgcgacgtcaagccctccaacatcctgctggacgagcggggccagatcaagttctgcgacttcggcatcagcggccgcctggtggactccaaagccaagacgcggagcgccggctgtgccgcctacatggcacccgagcgcattgaccccccagaccccaccaagccggactatgacatccgggccgacgtatggagcctgggcatctcgctggtggagctggcaacaggacagtttccctacaagaactgcaagacggactttgaggtcctcaccaaagtcctacaggaagagcccccgcttctgcccggacacatgggcttctcgggggacttccagtccttcgtcaaagactgccttactaaagatcacaggaagagaccaaagtataataagctacttgaacacagcttcatcaagcgctacgagacgctggaggtggacgtggcgtcctggttcaaggatgtcatggcgaagactgagtcaccgcggactagcggcgtcctgagccagccccacctgcccttcttcaggtag

SEQ ID NO: 21 - protein, 13 aa, Artificial Sequence
  Tag
MGSSHHHHHHSSG

SEQ ID NO: 22 - DNA, 1308 bp, Human
atggcggcgtcctccctggaacagaagctgtcccgcctggaagcaaagctgaagcaggagaaccgggaggcccggcggaggatcgacctcaacctggatatcagcccccagcggcccaggcccattattgtgatcactctaagccctgctcctgccccgtcccaacgagcagccctgcagctcccgctggccaacgatgggggcagccgctcgccatcctcagagagctccccgcagcaccccacgccccccgcccggccccgccacatgctggggctcccgtcaaccctgttcacaccccgcagcatggagagcattgagattgaccagaagctgcaggagatcatgaagcagacgggctacccgaccatcgggggccagcgctaccaggcagaaatcaacgacctggagaacttgggcgagatgggcagcggcacctgcggccaggtgtggaagatgcgcttccggaagaccggccacgtcattgccgttaagcaaatgcggcgctccgggaacaaggaggagaacaagcgcatcctcatggacctggatgtggtgctgaagagccacgactgcccctacatcgtgcagtgctttgggacgttcatcaccaacacggacgtcttcatcgccatggagctcatgggcacctgcgctgagaagctcaagaagcggatgcagggccccatccccgagcgcattctgggcaagatgacagtggcgattgtgaaggcgctgtactacctgaaagagaagcacggtgtcatccaccgcgacgtcaagccctccaacatcctgctggacgagcggggccagatcaagctctgcgacttcggcatcagcggccgcctggtggacgagaaagccaaggagcgggaggccggctgtgccgcctacatggcacccgagcgcattgaccccccagaccccaccaagccggactatgacatccgggccgacgtatggagcctgggcatctcgttggtggagctggcaacaggacagtttccctacaagaactgcaagacggactttgaggtcctcaccaaagtcctacaggaagagcccccgcttctgcccggacacatgggcttctcgggggacttccagtccttcgtcaaagactgccttactaaagatcacaggaagagaccaaagtataataagctacttgaacacagcttcatcaagcgctacgagacgctggaggtggacgtggcgtcctggttcaaggatgtcatggcgaagactgagtcaccgcggactagcggcgtcctgagccagccccacctgcccttcttcaggtag

SEQ ID NO: 23 - protein, 435 aa, Human
MAASSLEQKLSRLEAKLKQENREARRRIDLNLDISPQRPRPIIVITLSPAPAPSQRAALQLPLANDGGSRSPSSESSPQHPTPPARPRHMLGLPSTLFTPRSMESIEIDQKLQEIMKQTGYPTIGGQRYQAEINDLENLGEMGSGTCGQVWKMRFRKTGHVIAVKQMRRSGNKEENKRILMDLDVVLKSHDCPYIVQCFGTFITNTDVFIAMELMGTCAEKLKKRMQGPIPERILGKMTVAIVKALYYLKEKHGVIHRDVKPSNILLDERGQIKLCDFGISGRLVDEKAKEREAGCAAYMAPERIDPPDPTKPDYDIRADVWSLGISLVELATGQFPYKNCKTDFEVLTKVLQEEPPLLPGHMGFSGDFQSFVKDCLTKDHRKRPKYNKLLEHSFIKRYETLEVDVASWFKDVMAKTESPRTSGVLSQPHLPFFR